SEQUENCE LISTING (16 sequences)

SEQ ID NO: 1 - DNA, 3661 bp, Escherichia coli
  CDS (441)..(3623)
gaaaaaactgtgagtaagcgggtgataaatggtaaaagtcatcttgctataacaaggcttgcagtggaataatgaggccgtttccgtgtccatccttgttaaaacaagaaattttacggaataacccattttgcccgaccgatcatccacgcagcaatggcgtaagacgtattgatctttcaggcagttagcgggctgcgggttgcagtccttaccggtagatggaaatatttctggagagtaatacccagtctgtttcttgtataattgcgctgtttttccgcatgaaaaacgggcaaccgacactctgcgcctctttgagctgacgataaccgtgaggttggcgacgcgactagacacgaggccatcggttcacacccggaaaggcgttactttgcccgcagcttagtcgtcaatgtaagaataatgagtaagttacgatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagtcatcacgtttgtccgcgttgttctggtactggcaccgtgcgtgacaacgaatcgctgtcgctctctattctgcgtctgatcgaagaagaagcgctgaaagagaacacccaggaagttcacgccattgttcctgtgccaatcgcttcttacctgctgaatgaaaaacgttctgcggtaaatgccattgaaactcgtcaggacggtgtgcgctgtgtaattgtgccaaacgatcagatggaaaccccgcactaccacgtgctgcgcgtgcgtaaaggggaagaaaccccaaccttaagctacatgctgccgaagctgcatgaagaagcgatggcgctgccgtctgaagaagagttcgctgaacgtaagcgtccggaacaacctgcgctggcaacctttgccatgccggatgtgccgcctgcgccaacgccagctgaacctgccgcgcctgttgtagctccagcaccgaaagctgcaccggcaacaccagcagctcctgcacaacctgggctgttgagccgcttcttcggcgcactgaaagcgctgttcagcggtggtgaagaaaccaaaccgaccgagcaaccagcaccgaaagcagaagcgaaaccggaacgtcaacaggatcgtcgcaagcctcgtcagaacaaccgccgtgaccgtaatgagcgccgcgacacccgtagtgaacgtactgaaggcagcgataatcgcgaagaaaaccgtcgtaatcgtcgccaggcacagcagcagactgccgagacgcgtgagagccgtcagcaggctgaggtaacggaaaaagcgcgtaccgccgacgagcagcaagcgccgcgtcgtgaacgtagccgccgccgtaatgatgataaacgtcaggcgcaacaagaagcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgatcgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgccgctcctgcgcgtccgcaacctgttgagtaataattagctcaaagtaatcaagccctggtaactgc

SEQ ID NO: 2 - protein, 1061 aa, Escherichia coli
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVIDFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPALATFAMPDVPPAPTPAEPAAPVVAPAPKAAPATPAAPAQPGLLSRFFGALKALFSGGEETKPTEQPAPKAEAKPERQQDRRKPRQNNRRDRNERRDTRSERTEGSDNREENRRNRRQAQQQTAETRESRQQAEVTEKARTADEQQAPRRERSRRRNDDKRQAQQEAKALNVEEQSVQETEQEERVRPVQPRRKQRQLNQKVRYEQSVAEEAVVAPVVEETVAAEPIVQEAPAPRTELVKVPLPVVAQTAPEQQEENNADNRDNGGMPRRSRRSPRHLRVSGQRRRRYRDERYPTQSPMPLTVACASPELASGKVWIRYPIVRPQDVQVEEQREQEEVHVQPMVTEVPVAAAIEPVVSAPVVEEVAGVVEAPVQVAEPQPEVVETTHPEVIAAAVTEQPQVITESDVAVAQEVAEQAEPVVEPQEETADIEEVVETAEVVVAEPEVVAQPAAPVVAEVAAEVETVAAVEPEVTVEHNHATAPMTRAPAPEYVPEAPRHSDWQRPTFAFEGKGAAGGHTATHHASAAPARPQPVE

SEQ ID NO: 3 - DNA, 3532 bp, Artificial sequence
  E. coli RNase E deletion mutant
gaaaaaactgtgagtaagcgggtgataaatggtaaaagtcatcttgctataacaaggcttgcagtggaataatgaggccgtttccgtgtccatccttgttaaaacaagaaattttacggaataacccattttgcccgaccgatcatccacgcagcaatggcgtaagacgtattgatctttcaggcagttagcgggctgcgggttgcagtccttaccggtagatggaaatatttctggagagtaatacccagtctgtttcttgtataattgcgctgtttttccgcatgaaaaacgggcaaccgacactctgcgcctctttgagctgacgataaccgtgaggttggcgacgcgactagacacgaggccatcggttcacacccggaaaggcgttactttgcccgcagcttagtcgtcaatgtaagaataatgagtaagttacgatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagtcatcacgtttgtccgcgttgttctggtactggcaccgtgcgtgacaacgaatcgctgtcgctctctattctgcgtctgatcgaagaagaagcgctgaaagagaacacccaggaagttcacgccattgttcctgtgccaatcgcttcttacctgctgaatgaaaaacgttctgcggtaaatgccattgaaactcgtcaggacggtgtgcgctgtgtaattgtgccaaacgatcagatggaaaccccgcactaccacgtgctgcgcgtgcgtaaaggggaagaaaccccaaccttaagctacatgctgccgaagctgcatgaagaagcgatggcgctgccgtctgaagaagagttcgctgaacgtaagcgtccggaacaacctgcgctggcaacctttgccatgccggatgtgccgcctgcgccaacgccagctgaacctgccgcgcctgttgtagctccagcaccgaaagctgcaccggcaacaccagcagctcctgcacaacctgggctgttgagccgcttcttcggcgcactgaaagcgctgttcagcggtgaacgtactgaaggcagcgataatcgcgaagaaaaccgtcgtaatcgtcgccaggcacagcagcagactgccgagacgcgtgagagccgtcagcaggctgaggtaacggaaaaagcgcgtaccgccgacgagcagcaagcgccgcgtcgtgaacgtagccgccgccgtaatgatgataaacgtcaggcgcaacaagaagcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgatcgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgccgctcctgcgcgtccgcaacctgttgagtaataattagctcaaagtaatcaagccctggtaactgc

SEQ ID NO: 4 - protein, 1018 aa, Artificial sequence
  E. coli RNase E deletion mutant
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVIDFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPALATFAMPDVPPAPTPAEPAAPVVAPAPKAAPATPAAPAQPGLLSRFFGALKALFSGERTEGSDNREENRRNRRQAQQQTAETRESRQQAEVTEKARTADEQQAPRRERSRRRNDDKRQAQQEAKALNVEEQSVQETEQEERVRPVQPRRKQRQLNQKVRYEQSVAEEAVVAPVVEETVAAEPIVQEAPAPRTELVKVPLPVVAQTAPEQQEENNADNRDNGGMPRRSRRSPRHLRVSGQRRRRYRDERYPTQSPMPLTVACASPELASGKVWIRYPIVRPQDVQVEEQREQEEVHVQPMVTEVPVAAAIEPVVSAPVVEEVAGVVEAPVQVAEPQPEVVETTHPEVIAAAVTEQPQVITESDVAVAQEVAEQAEPVVEPQEETADIEEVVETAEVVVAEPEVVAQPAAPVVAEVAAEVETVAAVEPEVTVEHNHATAPMTRAPAPEYVPEAPRHSDWQRPTFAFEGKGAAGGHTATHHASAAPARPQPVE

SEQ ID NO: 5 - DNA, 3334 bp, Artificial sequence
  E. coli RNase E deletion mutant
gaaaaaactgtgagtaagcgggtgataaatggtaaaagtcatcttgctataacaaggcttgcagtggaataatgaggccgtttccgtgtccatccttgttaaaacaagaaattttacggaataacccattttgcccgaccgatcatccacgcagcaatggcgtaagacgtattgatctttcaggcagttagcgggctgcgggttgcagtccttaccggtagatggaaatatttctggagagtaatacccagtctgtttcttgtataattgcgctgtttttccgcatgaaaaacgggcaaccgacactctgcgcctctttgagctgacgataaccgtgaggttggcgacgcgactagacacgaggccatcggttcacacccggaaaggcgttactttgcccgcagcttagtcgtcaatgtaagaataatgagtaagttacgatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagtcatcacgtttgtccgcgttgttctggtactggcaccgtgcgtgacaacgaatcgctgtcgctctctattctgcgtctgatcgaagaagaagcgctgaaagagaacacccaggaagttcacgccattgttcctgtgccaatcgcttcttacctgctgaatgaaaaacgttctgcggtaaatgccattgaaactcgtcaggacggtgtgcgctgtgtaattgtgccaaacgatcagatggaaaccccgcactaccacgtgctgcgcgtgcgtaaaggggaagaaaccccaaccttaagctacatgctgccgaagctgcatgaagaagcgatggcgctgccgtctgaagaagagttcgctgaacgtaagcgtccggaacaacctgcgctggcaacctttgccatgccggatgtgccgcctgcgccaacgccagctgaacctgccgcgcctgttgtagctccagcaccgaaagctgcaccggcaacaccagcagctcctgcacaacctgggctgttgagccgcttcttcggcgcactgaaagcgctgttcagcggtgcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgatcgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgccgctcctgcgcgtccgcaacctgttgagtaataattagctcaaagtaatcaagccctggtaactgc

SEQ ID NO: 6 - protein, 952 aa, Artificial sequence
  E. coli RNase E deletion mutant
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVIDFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPALATFAMPDVPPAPTPAEPAAPVVAPAPKAAPATPAAPAQPGLLSRFFGALKALFSGAKALNVEEQSVQETEQEERVRPVQPRRKQRQLNQKVRYEQSVAEEAVVAPVVEETVAAEPIVQEAPAPRTELVKVPLPVVAQTAPEQQEENNADNRDNGGMPRRSRRSPRHLRVSGQRRRRYRDERYPTQSPMPLTVACASPELASGKVWIRYPIVRPQDVQVEEQREQEEVHVQPMVTEVPVAAAIEPVVSAPVVEEVAGVVEAPVQVAEPQPEVVETTHPEVIAAAVTEQPQVITESDVAVAQEVAEQAEPVVEPQEETADIEEVVETAEVVVAEPEVVAQPAAPVVAEVAAEVETVAAVEPEVTVEHNHATAPMTRAPAPEYVPEAPRHSDWQRPTFAFEGKGAAGGHTATHHASAAPARPQPVE

SEQ ID NO: 7 - DNA, 3388 bp, Artificial sequence
  E. coli RNase E deletion mutant
gaaaaaactgtgagtaagcgggtgataaatggtaaaagtcatcttgctataacaaggcttgcagtggaataatgaggccgtttccgtgtccatccttgttaaaacaagaaattttacggaataacccattttgcccgaccgatcatccacgcagcaatggcgtaagacgtattgatctttcaggcagttagcgggctgcgggttgcagtccttaccggtagatggaaatatttctggagagtaatacccagtctgtttcttgtataattgcgctgtttttccgcatgaaaaacgggcaaccgacactctgcgcctctttgagctgacgataaccgtgaggttggcgacgcgactagacacgaggccatcggttcacacccggaaaggcgttactttgcccgcagcttagtcgtcaatgtaagaataatgagtaagttacgatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagtcatcacgtttgtccgcgttgttctggtactggcaccgtgcgtgacaacgaatcgctgtcgctctctattctgcgtctgatcgaagaagaagcgctgaaagagaacacccaggaagttcacgccattgttcctgtgccaatcgcttcttacctgctgaatgaaaaacgttctgcggtaaatgccattgaaactcgtcaggacggtgtgcgctgtgtaattgtgccaaacgatcagatggaaaccccgcactaccacgtgctgcgcgtgcgtaaaggggaagaaaccccaaccttaagctacatgctgccgaagctgcatgaagaagcgatggcgctgccgtctgaagaagagttcgctgaacgtaagcgtccggaacaacctgcgctggcaacctttgccatgccggatgtgccgcctgcgccaacgccagctgaacctgccgcgcctgttgtagctccagcaccgaaagctgcaccggcaacaccagcagctcctgcacaacctgggctgttgagccgcttcttcggcgcactgaaagcgctgttcagcggtggtgaagaaaccaaaccgaccgagcaaccagcaccgaaagcagaagcgaaaccggcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgatcgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgccgctcctgcgcgtccgcaacctgttgagtaataattagctcaaagtaatcaagccctggtaactgc

SEQ ID NO: 8 - protein, 970 aa, Artificial sequence
  E. coli RNase E deletion mutant
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVIDFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPALATFAMPDVPPAPTPAEPAAPVVAPAPKAAPATPAAPAQPGLLSRFFGALKALFSGGEETKPTEQPAPKAEAKPAKALNVEEQSVQETEQEERVRPVQPRRKQRQLNQKVRYEQSVAEEAVVAPVVEETVAAEPIVQEAPAPRTELVKVPLPVVAQTAPEQQEENNADNRDNGGMPRRSRRSPRHLRVSGQRRRRYRDERYPTQSPMPLTVACASPELASGKVWIRYPIVRPQDVQVEEQREQEEVHVQPMVTEVPVAAAIEPVVSAPVVEEVAGVVEAPVQVAEPQPEVVETTHPEVIAAAVTEQPQVITESDVAVAQEVAEQAEPVVEPQEETADIEEVVETAEVVVAEPEVVAQPAAPVVAEVAAEVETVAAVEPEVTVEHNHATAPMTRAPAPEYVPEAPRHSDWQRPTFAFEGKGAAGGHTATHHASAAPARPQPVE

SEQ ID NO: 9 - DNA, 3586 bp, Artificial sequence
  E. coli RNase E deletion mutant
gaaaaaactgtgagtaagcgggtgataaatggtaaaagtcatcttgctataacaaggcttgcagtggaataatgaggccgtttccgtgtccatccttgttaaaacaagaaattttacggaataacccattttgcccgaccgatcatccacgcagcaatggcgtaagacgtattgatctttcaggcagttagcgggctgcgggttgcagtccttaccggtagatggaaatatttctggagagtaatacccagtctgtttcttgtataattgcgctgtttttccgcatgaaaaacgggcaaccgacactctgcgcctctttgagctgacgataaccgtgaggttggcgacgcgactagacacgaggccatcggttcacacccggaaaggcgttactttgcccgcagcttagtcgtcaatgtaagaataatgagtaagttacgatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagtcatcacgtttgtccgcgttgttctggtactggcaccgtgcgtgacaacgaatcgctgtcgctctctattctgcgtctgatcgaagaagaagcgctgaaagagaacacccaggaagttcacgccattgttcctgtgccaatcgcttcttacctgctgaatgaaaaacgttctgcggtaaatgccattgaaactcgtcaggacggtgtgcgctgtgtaattgtgccaaacgatcagatggaaaccccgcactaccacgtgctgcgcgtgcgtaaaggggaagaaaccccaaccttaagctacatgctgccgaagctgcatgaagaagcgatggcgctgccgtctgaagaagagttcgctgaacgtaagcgtccggaacaacctgcgctggcaacctttgccatgccggatgtgccgcctgcgccaacgccagctgaacctgccgcgcctgttgtagctccagcaccgaaagctgcaccggcaacaccagcagctcctgcacaacctgggctgttgagccgcttcttcggcgcactgaaagcgctgttcagcggtggtgaagaaaccaaaccgaccgagcaaccagcaccgaaagcagaagcgaaaccggaacgtactgaaggcagcgataatcgcgaagaaaaccgtcgtaatcgtcgccaggcacagcagcagactgccgagacgcgtgagagccgtcagcaggctgaggtaacggaaaaagcgcgtaccgccgacgagcagcaagcgccgcgtcgtgaacgtagccgccgccgtaatgatgataaacgtcaggcgcaacaagaagcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgatcgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgccgctcctgcgcgtccgcaacctgttgagtaataattagctcaaagtaatcaagccctggtaactgc

SEQ ID NO: 10 - protein, 1036 aa, Artificial sequence
  E. coli RNase E deletion mutant
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVIDFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPALATFAMPDVPPAPTPAEPAAPVVAPAPKAAPATPAAPAQPGLLSRFFGALKALFSGGEETKPTEQPAPKAEAKPERTEGSDNREENRRNRRQAQQQTAETRESRQQAEVTEKARTADEQQAPRRERSRRRNDDKRQAQQEAKALNVEEQSVQETEQEERVRPVQPRRKQRQLNQKVRYEQSVAEEAVVAPVVEETVAAEPIVQEAPAPRTELVKVPLPVVAQTAPEQQEENNADNRDNGGMPRRSRRSPRHLRVSGQRRRRYRDERYPTQSPMPLTVACASPELASGKVWIRYPIVRPQDVQVEEQREQEEVHVQPMVTEVPVAAAIEPVVSAPVVEEVAGVVEAPVQVAEPQPEVVETTHPEVIAAAVTEQPQVITESDVAVAQEVAEQAEPVVEPQEETADIEEVVETAEVVVAEPEVVAQPAAPVVAEVAAEVETVAAVEPEVTVEHNHATAPMTRAPAPEYVPEAPRHSDWQRPTFAFEGKGAAGGHTATHHASAAPARPQPVE

SEQ ID NO: 11 - DNA, 3487 bp, Artificial sequence
  E. coli RNase E deletion mutant
gaaaaaactgtgagtaagcgggtgataaatggtaaaagtcatcttgctataacaaggcttgcagtggaataatgaggccgtttccgtgtccatccttgttaaaacaagaaattttacggaataacccattttgcccgaccgatcatccacgcagcaatggcgtaagacgtattgatctttcaggcagttagcgggctgcgggttgcagtccttaccggtagatggaaatatttctggagagtaatacccagtctgtttcttgtataattgcgctgtttttccgcatgaaaaacgggcaaccgacactctgcgcctctttgagctgacgataaccgtgaggttggcgacgcgactagacacgaggccatcggttcacacccggaaaggcgttactttgcccgcagcttagtcgtcaatgtaagaataatgagtaagttacgatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagtcatcacgtttgtccgcgttgttctggtactggcaccgtgcgtgacaacgaatcgctgtcgctctctattctgcgtctgatcgaagaagaagcgctgaaagagaacacccaggaagttcacgccattgttcctgtgccaatcgcttcttacctgctgaatgaaaaacgttctgcggtaaatgccattgaaactcgtcaggacggtgtgcgctgtgtaattgtgccaaacgatcagatggaaaccccgcactaccacgtgctgcgcgtgcgtaaaggggaagaaaccccaaccttaagctacatgctgccgaagctgcatgaagaagcgatggcgctgccgtctgaagaagagttcgctgaacgtaagcgtccggaacaacctgcgctggcaacctttgccatgccggatgtgccgcctgcgccaacgccagctgaacctgccgcgcctgttgtagctccagcaccgaaagctgcaccggcaacaccagcagctcctgcacaacctgggctgttgagccgcttcttcggcgcactgaaagcgctgttcagcggtggtgaagaaaccaaaccgaccgagcaaccagcaccgaaagcagaagcgaaaccggaacgtcaacaggatcgtcgcaagcctcgtcagaacaaccgccgtgaccgtaatgagcgccgcgacacccgtagtgaacgtactgaaggcagcgataatgcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgatcgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgccgctcctgcgcgtccgcaacctgttgagtaataattagctcaaagtaatcaagccctggtaactgc

SEQ ID NO: 12 - protein, 1003 aa, Artificial sequence
  E. coli RNase E deletion mutant
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVIDFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPALATFAMPDVPPAPTPAEPAAPVVAPAPKAAPATPAAPAQPGLLSRFFGALKALFSGGEETKPTEQPAPKAEAKPERQQDRRKPRQNNRRDRNERRDTRSERTEGSDNAKALNVEEQSVQETEQEERVRPVQPRRKQRQLNQKVRYEQSVAEEAVVAPVVEETVAAEPIVQEAPAPRTELVKVPLPVVAQTAPEQQEENNADNRDNGGMPRRSRRSPRHLRVSGQRRRRYRDERYPTQSPMPLTVACASPELASGKVWIRYPIVRPQDVQVEEQREQEEVHVQPMVTEVPVAAAIEPVVSAPVVEEVAGVVEAPVQVAEPQPEVVETTHPEVIAAAVTEQPQVITESDVAVAQEVAEQAEPVVEPQEETADIEEVVETAEVVVAEPEVVAQPAAPVVAEVAAEVETVAAVEPEVTVEHNHATAPMTRAPAPEYVPEAPRHSDWQRPTFAFEGKGAAGGHTATHHASAAPARPQPVE

SEQ ID NO: 13 - DNA, 3031 bp, Artificial sequence
  E. coli RNase E deletion mutant
gaaaaaactgtgagtaagcgggtgataaatggtaaaagtcatcttgctataacaaggcttgcagtggaataatgaggccgtttccgtgtccatccttgttaaaacaagaaattttacggaataacccattttgcccgaccgatcatccacgcagcaatggcgtaagacgtattgatctttcaggcagttagcgggctgcgggttgcagtccttaccggtagatggaaatatttctggagagtaatacccagtctgtttcttgtataattgcgctgtttttccgcatgaaaaacgggcaaccgacactctgcgcctctttgagctgacgataaccgtgaggttggcgacgcgactagacacgaggccatcggttcacacccggaaaggcgttactttgcccgcagcttagtcgtcaatgtaagaataatgagtaagttacgatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagtcatcacgtttgtccgcgttgttctggtactggcaccgtgcgtgacaacgaatcgctgtcgctctctattctgcgtctgatcgaagaagaagcgctgaaagagaacacccaggaagttcacgccattgttcctgtgccaatcgcttcttacctgctgaatgaaaaacgttctgcggtaaatgccattgaaactcgtcaggacggtgtgcgctgtgtaattgtgccaaacgatcagatggaaaccccgcactaccacgtgctgcgcgtgcgtaaaggggaagaaaccccaaccttaagctacatgctgccgaagctgcatgaagaagcgatggcgctgccgtctgaagaagagttcgctgaacgtaagcgtccggaacaacctgcgctggcaacctttgccatgccggatgtgccgcctgcgccaacgccagctgaacctgccgcgcctgttgtagctccagcaccgaaagctgcaccggcaacaccagcagctcctgcacaacctgggctgttgagccgcttcttcggcgcactgaaagcgctgttcagcggtggtgaagaaaccaaaccgaccgagcaaccagcaccgaaagcagaagcgaaaccggaacgtcaacaggatcgtcgcaagcctcgtcagaacaaccgccgtgaccgtaatgagcgccgcgacacccgtagtgaacgtactgaaggcagcgataattatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgatcgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgccgctcctgcgcgtccgcaacctgttgagtaataattagctcaaagtaatcaagccctggtaactgc

SEQ ID NO: 14 - protein, 851 aa, Artificial sequence
  E. coli RNase E deletion mutant
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVIDFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPALATFAMPDVPPAPTPAEPAAPVVAPAPKAAPATPAAPAQPGLLSRFFGALKALFSGGEETKPTEQPAPKAEAKPERQQDRRKPRQNNRRDRNERRDTRSERTEGSDNYPIVRPQDVQVEEQREQEEVHVQPMVTEVPVAAAIEPVVSAPVVEEVAGVVEAPVQVAEPQPEVVETTHPEVIAAAVTEQPQVITESDVAVAQEVAEQAEPVVEPQEETADIEEVVETAEVVVAEPEVVAQPAAPVVAEVAAEVETVAAVEPEVTVEHNHATAPMTRAPAPEYVPEAPRHSDWQRPTFAFEGKGAAGGHTATHHASAAPARPQPVE

SEQ ID NO: 15 - DNA, 3307 bp, Artificial sequence
  E. coli RNase E deletion mutant
gaaaaaactgtgagtaagcgggtgataaatggtaaaagtcatcttgctataacaaggcttgcagtggaataatgaggccgtttccgtgtccatccttgttaaaacaagaaattttacggaataacccattttgcccgaccgatcatccacgcagcaatggcgtaagacgtattgatctttcaggcagttagcgggctgcgggttgcagtccttaccggtagatggaaatatttctggagagtaatacccagtctgtttcttgtataattgcgctgtttttccgcatgaaaaacgggcaaccgacactctgcgcctctttgagctgacgataaccgtgaggttggcgacgcgactagacacgaggccatcggttcacacccggaaaggcgttactttgcccgcagcttagtcgtcaatgtaagaataatgagtaagttacgatgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagtcatcacgtttgtccgcgttgttctggtactggcaccgtgcgtgacaacgaatcgctgtcgctctctattctgcgtctgatcgaagaagaagcgctgaaagagaacacccaggaagttcacgccattgttcctgtgccaatcgcttcttacctgctgaatgaaaaacgttctgcggtaaatgccattgaaactcgtcaggacggtgtgcgctgtgtaattgtgccaaacgatcagatggaaaccccgcactaccacgtgctgcgcgtgcgtaaaggggaagaaaccccaaccttaagctacatgctgccgaagctgcatgaagaagcgatggcgctgccgtctgaagaagagttcgctgaacgtaagcgtccggaacaacctgcgctggcaacctttgccatgccggatgtgccgcctgcgccaacgccagctgaacctgccgcgcctgttgtagctccagcaccgaaagctgcaccggcaacaccagcagctcctgcacaacctgggctgttgagccgcttcttcggcgcactgaaagcgctgttcagcggtggtgaagaaaccaaaccgaccgagcaaccagcaccgaaagcagaagcgaaaccggaacgtcaacaggatcgtcgcaagcctcgtcagaacaaccgccgtgaccgtaatgagcgccgcgacacccgtagtgaacgtactgaaggcagcgataatcgcgaagaaaaccgtcgtaatcgtcgccaggcacagcagcagactgccgagacgcgtgagagccgtcagcaggctgaggtaacggaaaaagcgcgtaccgccgacgagcagcaagcgccgcgtcgtgaacgtagccgccgccgtaatgatgataaacgtcaggcgcaacaagaagcgaaggcgctgaatgttgaagagcaatctgttcaggaaaccgaacaggaagaacgtgtacgtccggttcagccgcgtcgtaaacagcgtcagctcaatcagtatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgatcgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgccgctcctgcgcgtccgcaacctgttgagtaataattagctcaaagtaatcaagccctggtaactgc

SEQ ID NO: 16 - protein, 943 aa, Artificial sequence
  E. coli RNase E deletion mutant
MKRMLINATQQEELRVALVDGQRLYDLDIESPGHEQKKANIYKGKITRIEPSLEAAFVDYGAERHGFLPLKEIAREYFPANYSAHGRPNIKDVLREGQEVIVQIDKEERGNKGAALTTFISLAGSYLVLMPNNPRAGGISRRIEGDDRTELKEALASLELPEGMGLIVRTAGVGKSAEALQWDLSFRLKHWEAIKKAAESRPAPFLIHQESNVIVRAFRDYLRQDIGEILIDNPKVLELARQHIAALGRPDFSSKIKLYTGEIPLFSHYQIESQIESAFQREVRLPSGGSIVIDSTEALTAIDINSARATRGGDIEETAFNTNLEAADEIARQLRLRDLGGLIVIDFIDMTPVRHQRAVENRLREAVRQDRARIQISHISRFGLLEMSRQRLSPSLGESSHHVCPRCSGTGTVRDNESLSLSILRLIEEEALKENTQEVHAIVPVPIASYLLNEKRSAVNAIETRQDGVRCVIVPNDQMETPHYHVLRVRKGEETPTLSYMLPKLHEEAMALPSEEEFAERKRPEQPALATFAMPDVPPAPTPAEPAAPVVAPAPKAAPATPAAPAQPGLLSRFFGALKALFSGGEETKPTEQPAPKAEAKPERQQDRRKPRQNNRRDRNERRDTRSERTEGSDNREENRRNRRQAQQQTAETRESRQQAEVTEKARTADEQQAPRRERSRRRNDDKRQAQQEAKALNVEEQSVQETEQEERVRPVQPRRKQRQLNQYPIVRPQDVQVEEQREQEEVHVQPMVTEVPVAAAIEPVVSAPVVEEVAGVVEAPVQVAEPQPEVVETTHPEVIAAAVTEQPQVITESDVAVAQEVAEQAEPVVEPQEETADIEEVVETAEVVVAEPEVVAQPAAPVVAEVAAEVETVAAVEPEVTVEHNHATAPMTRAPAPEYVPEAPRHSDWQRPTFAFEGKGAAGGHTATHHASAAPARPQPVE

(19) United States
(12) Patent Application Publication
(10) Pub. No.: US 2004/0126842 A1
(43) Pub. Date: Jul. 1, 2004

(54) Mutant E. coli strains, and their use for producing recombinant polypeptides

(76) Inventors: Marc Dreyfus, Paris (FR); Pascal Lopez, Saint-Laurent-du-Pont (FR); Agamemnon J. Carpousis, Romainville Sainte Agne (FR)

Correspondence Address: YOUNG & THOMPSON, 745 SOUTH 23RD STREET 2ND FLOOR, ARLINGTON, VA, 22202

(21) Appl. No.: 10/655,042

(22) Filed: Sep. 5, 2003

Related U.S. Application Data
(63) Continuation-in-part of application No. 09/762,481, filed on May 17, 2001, now Pat. No. 6,632,639.
Application No. 09/762,481 is a 371 of international application No. PCT/FR99/01879, filed on Jul. 29, 1999.

(30) Foreign Application Priority Data
Aug. 7, 1998 (FR) 98/10197

(51) Int. Cl. C12P 21/06

(52) U.S. Cl. 435/69.1; 435/243; 536/23.2

(57) ABSTRACT

A process for producing predetermined recombinant polypeptides or proteins, comprising expressing the polypeptides or proteins in Escherichia coli (E. coli) strains whose gene coding RNase E comprises a mutation such that the enzyme produced upon expression of this mutated gene exhibits reduced activity for degrading the messenger RNA (m-RNA) encoding the polypeptides or proteins, compared to bulk mRNA, the mutation not significantly affecting growth of the E. coli strains, and wherein the mutation corresponds to the substitution or deletion of one up to all the nucleotides located in the region delimited by the nucleotide at position 2193 and the nucleotide at position 2975 of the DNA sequence coding the RNase E represented by SEQ ID NO: 1.

This application is a continuation-in-part of U.S. patent application Ser. No. 09/762,481, filed on Feb. 7, 2001. Application Ser. No. 09/762,481 is the National Phase of PCT International Application No. PCT/FR99/01879 filed on Jul. 9, 1999.

The invention concerns certain mutant E. coli strains, and their use for performing processes for producing recombinant polypeptides.

Genomic study of higher organisms, micro-organisms, and viruses almost invariably requires, in addition to the cloning of their genes, large-scale production of their products (proteins), so as for example to obtain antibodies or to perform biochemical or crystallographic studies.

From the applications viewpoint, the utilization in the medical field of numerous human peptides and proteins also requires expression of corresponding genes in heterologous organisms.

Although expression systems have been established in various eukaryotic hosts (especially in yeasts, insects and primate cells), the most widely used host for these expression strategies remains the bacteria Escherichia coli (E. coli). The list of proteins of biotechnological or pharmacological interest that are produced in E. coli is extensive; classic examples include human insulin and human growth hormone.

The most well-known expression system in prokaryotes was developed in the USA by the Studier and Richardson groups, during the 1980's (Tabor and Richardson, 1985; Studier and Moffat, 1986). It is based on exploiting the properties of T7 RNA polymerase (namely RNA polymerase encoded by the T7 bacteriophage). That enzyme, which can be expressed in E. coli cells without toxicity, recognizes a very specific promoter. Any gene of interest (target gene) may be transcribed very efficiently, upon placing it downstream of this promoter and introducing it into an E. coli cell expressing T7 polymerase.

Nevertheless, in terms of expression, the results remain uncertain. Some target genes may be duly overexpressed, whereas others are expressed only moderately or not at all.

Previous work by the inventors revealed that one of the principal causes of these setbacks resides in the specific instability of the m-RNA synthesized by T7 RNA polymerase, which causes a decrease in the number of polypeptides synthesized per message (Lopez et al., 1994; Iost and Dreyfus, 1994, 1995). This instability is the consequence of the high speed of elongation of T7 RNA polymerase (Makarova et al., 1995). Specifically, the elongation speed of T7 polymerase, in contrast to that of bacterial RNA polymerase, is much greater than the translation speed of m-RNA by ribosomes. Nascent m-RNA is therefore exposed over most of its length, and is therefore readily attacked by nucleases, and in E. coli especially by the E-type ribonuclease (or RNase E), whose amino acid sequence is described by Casaregola et al. (Casaregola et al., 1992, 1994).

RNase E is an essential enzyme of E. coli; it is involved both in the degradation of m-RNA, and in the maturation of ribosomal RNA (rRNA) and transfer RNA (tRNA). Mutations in the catalytic region (that is, in the N-terminal portion of RNase E) affect these functions at the same time, and slow down or even arrest the growth of E. coli (Cohen and McDowall, 1997).

On the other hand, deletions in the C-terminal portion of RNase E do not affect the viability of E. coli. Specifically, by searching for revertants of mutations in a protein (MukB) necessary for the segregation of chromosomes after replication, Kido et al. obtained various viable mutations in the rne gene, coding RNase E in E. coli, which cause synthesis of an RNase E that is truncated in its C-terminal portion (Kido et al., 1996). These authors concluded from these experiments that the C-terminal portion of RNase E is not essential for viability of E. coli. They moreover formed the hypothesis that suppression of the mukB mutations by truncating of the RNase E, reflects the fact that truncated RNase E is less effective than the wild-type enzyme for degrading mukB m-RNA. Thus stabilized, a stronger synthesis of the mutant MukB protein could be achieved, thereby correcting the phenotype associated with the mutation. However, this stabilization of the mukB messenger was not demonstrated, and other authors proposed an entirely different interpretation to explain the suppressive effect of the truncating of RNase E on mukB mutations (Cohen and McDowall, 1997). These authors postulate in particular a direct interaction between RNase E and MukB. The basis for that idea is the fact that RNase E has a very substantial similarity with eukaryotic myosin (Casaregola et al., 1992: McDowall et al., 1993), which suggests that aside from its own RNase activity, it could, like MukB, play a structural role.

The present invention arises from the demonstration by the inventors of the fact that the truncating of RNase E causes an overall stabilization of cellular m-RNA, considered as a whole, as well as of the majority of individual m-RNAs that were examined, without significantly impeding the maturation of the r-RNAs (Lopez et al., 1999).

In that regard, the effect of the deletion is very different from that of a mutation in the N-terminal region, such as the ams mutation (Ono and Kuwano, 1979), renamed rne1 (Babitzke and Kushner, 1991), which confers thermosensitive activity to RNase E. For example, at 37° C., this latter mutation causes a moderate increase in the lifespan of the m-RNAs (1.5 times each on average; the lifespan of the m-RNAs is here defined as the time during which they serve as a matrix for protein synthesis (Mudd et al., 1990a)), but it also causes a significant slowdown in maturation of the r-RNAs (estimated by the “Northern” method; see Lopez et al., 1994) and it retards the growth by a factor of 2. On the contrary, deletion of the C-terminal portion of RNase E, especially of amino acids 586 to 1061 of this latter, causes a more significant stabilization of the m-RNA (two times on average), without causing a slowdown in the maturation of the r-RNA and without retarding growth. Thus, in hindsight, it is likely that the lack of growth that was observed with N-terminal mutations of RNase E, is due solely to the inability of the cells to mature r-RNA (or tRNA), and not to the slowing of mRNA degradation.

In summary, deletions in the C-terminal portion of RNase E have no effect on the activity of the catalytic domain, judging from the rapid maturation of the r-RNA. That rapid maturation explains why the cells containing such a deletion are viable. On the other hand, the deletion stabilizes the m-RNA as a whole, perhaps because it inhibits the association of the RNase E with other enzymes within a multi-protein structure, the so-called “degradosome”, which might be necessary for degradation of the m-RNA (Carpousis et al., 1994; Miczack et al, 1996; Py et al., 1996; Kido et al., 1996; Cohen and McDowall, 1997). The important point from the perspective of the invention is that, by virtue of these deletions, it is possible to obtain E. coli strains having enhanced m-RNA stability, while preserving normal growth.

The inventors have also shown that the stabilization of m-RNA due to the deletion of the C-terminal portion of RNase E, is not uniform, but rather is more pronounced for less stable m-RNA. As is known, this is often the case for the m-RNA of “target” genes in expression systems, particularly in systems based on T7 RNA polymerase activity. The contribution of these m-RNAs to the overall protein synthesis is therefore enhanced by the presence of the deletion. E. coli strains comprising such a deletion therefore express recombinant exogenous polypeptides with sharply higher yields (in particular about 3 to 25 times higher) with respect to the expression yields of those recombinant polypeptides by E. coli strains not comprising that mutation, especially when the expression of the said recombinant polypeptides is placed under the control of T7 RNA polymerase.

The present invention therefore has as an object to provide novel processes for producing recombinant proteins or polypeptides from E. coli, especially those of pharmaceutical or biological interest, at production yields substantially greater than those of the processes described up to now.

The present invention also has as an object to provide novel E. coli strains for operating the above-mentioned processes, as well as methods for preparing such strains.

The present invention relates to a process for producing predetermined recombinant polypeptides or proteins, comprising expressing said polypeptides or proteins in Escherichia coli (E. coli) strains whose gene coding RNase E comprises a mutation such that the enzyme produced upon expression of this mutated gene exhibits reduced activity for degrading the messenger RNA (m-RNA) encoding said polypeptides or proteins, when compared to bulk mRNA (i.e. the E. coli endogen cellular mRNAs on which said enzymes produced by said mutated RNase E coding genes have no or little reduced activity of degradation), said mutation not significantly affecting growth of the said E. coli strains, and wherein said mutation corresponds to the substitution or deletion of one up to all the nucleotides located in the region delimited by the nucleotide at position 2193 and the nucleotide at position 2975 of the DNA sequence coding the RNase E represented by SEQ ID NO: 1.

The present invention more particularly concerns a process as described above wherein the gene coding RNase E of said E. coli strains comprises a mutation such that the enzyme produced upon expression of this mutated gene preserves the maturation activity of the r-RNA of the RNase E, but exhibits reduced activity for degrading the messenger RNA (m-RNA) encoding said recombinant polypeptides or proteins, compared to bulk mRNA.

The invention relates more particularly to a process as defined above, characterized in that the mutation causes the deletion of at least one, up to all, of the amino acids at position 585 to 845 of the sequence of RNase E represented by SEQ ID NO: 2.

The invention yet more particularly concerns a process as defined above, wherein said mutation corresponds to the deletion

of the DNA fragment delimited by the nucleotides at positions 2193 to 2321 of SEQ ID NO: 1, thus leading to a mutated RNase E gene represented by SEQ ID NO: 3 and coding for the mutated RNase E protein RneΔ24 represented by SEQ ID NO: 4 and corresponding to SEQ ID NO: 2 wherein the sequence delimited by the aminoacids at positions 585 to 627 is deleted,

of the DNA fragment delimited by the nucleotides at positions 2193 to 2519 of SEQ ID NO: 1, thus leading to a mutated RNase E gene represented by SEQ ID NO: 5 and coding for the mutated RNase E protein RneΔ23 represented by SEQ ID NO: 6 and corresponding to SEQ ID NO: 2 wherein the sequence delimited by the aminoacids at positions 585 to 693 is deleted,

of the DNA fragment delimited by the nucleotides at positions 2247 to 2519 of SEQ ID NO: 1, thus leading to a mutated RNase E gene represented by SEQ ID NO: 7 and coding for the mutated RNase E protein RneΔ22 represented by SEQ ID NO: 8 and corresponding to SEQ ID NO : 2 wherein the sequence delimited by the aminoacids at positions 603 to 693 is deleted,

of the DNA fragment delimited by the nucleotides at positions 2247 to 2321 of SEQ ID NO: 1, thus leading to a mutated RNase E gene represented by SEQ ID NO: 9 and coding for the mutated RNase E protein RneΔ21 represented by SEQ ID NO: 4 and corresponding to SEQ ID NO: 2 wherein the sequence delimited by the aminoacids at positions 603 to 627 is deleted,

of the DNA fragment delimited by the nucleotides at positions 2346 to 2519 of SEQ ID NO: 1, thus leading to a mutated RNase E gene represented by SEQ ID NO: 11 and coding for the mutated RNase E protein RneΔ17 represented by SEQ ID NO: 12 and corresponding to SEQ ID NO: 2 wherein the sequence delimited by the aminoacids at positions 636 to 693 is deleted,

of the DNA fragment delimited by the nucleotides at positions 2346 to 2975 of SEQ ID NO: 1, thus leading to a mutated RNase E gene represented by SEQ ID NO: 13 and coding for the mutated RNase E protein RneΔ14 represented by SEQ ID NO: 14 and corresponding to SEQ ID NO: 2 wherein the sequence delimited by the aminoacids at positions 636 to 845 is deleted,

of the DNA fragment delimited by the nucleotides at positions 2622 to 2975 of SEQ ID NO: 1, thus leading to a mutated RNase E gene represented by SEQ ID NO: 15 and coding for the mutated RNase E protein RneΔ18 represented by SEQ ID NO: 16 and corresponding to SEQ ID NO: 2 wherein the sequence delimited by the aminoacids at positions 728 to 845 is deleted.

Advantageously, the above-mentioned mutant E. coli strains, used in the context of the invention, contain an exogenous inducible expression system, under the control of which is placed the expression of predetermined recombinant polypeptides, especially the inducible expression system using RNA polymerase of the T7 bacteriophage.

The invention also relates to a process for producing predetermined recombinant polypeptides as defined above, characterized in that it comprises:

a step of transforming E. coli strains whose gene coding RNase E comprises a mutation as defined above such that enzyme produced upon expression of this mutated gene possesses reduced degradation activity for m-RNA, compared to bulk mRNA, this mutation not significantly affecting growth of the said E. coli strains, with a vector, especially a plasmid, containing the nucleotide sequence coding one or several recombinant polypeptides,

culturing the transformed E. coli strains obtained in the preceding step, for a time sufficient to permit expression of the recombinant polypeptide or polypeptides in the E. coli cells,

and recovery of the recombinant polypeptide or polypeptides produced during the preceding step, optionally after purification of these latter, especially by chromatography, electrophoresis, or selective precipitation.

The invention more particularly has as an object any process for producing predetermined recombinant polypeptides, as defined above, characterized in that it comprises:

a step of transforming E. coli strains as described above, with a vector, especially a plasmid, containing the nucleotide sequence coding one or several recombinant polypeptides, so as to obtain the above-mentioned E. coli strains, in which transcription of the said nucleotide sequence coding one or several recombinant polypeptides is placed under the control of an inducible expression system,

culturing the transformed E. coli strains obtained during the preceding step, and inducing the said expression system, for a time sufficient to permit expression of the recombinant polypeptide or polypeptides in E. coli cells, the inducing of the said expression system especially being effected by causing synthesis of T7 RNA polymerase when the said expression system involves that polymerase; this synthesis may notably be provoked by adding IPTG to the culture medium, or by raising the temperature, when the gene coding for this RNA polymerase is placed under the control of a promoter regulated by the lac repressor (Studier and Moffat, 1986), or under the control of a thermo-inducible promoter (Tabor and Richardson, 1985),

and recovering the recombinant polypeptide or polypeptides produced during the preceding step.

The invention also concerns E. coli strains transformed such that they contain an inducible expression system, and whose gene coding RNase E comprises a mutation such that the enzyme produced upon expression of this mutated gene exhibits reduced activity for degrading the messenger RNA (m-RNA) encoding said polypeptides or proteins, compared to bulk mRNA, this mutation not significantly affecting growth of the said E. coli strains, and wherein said mutation corresponds to the deletion :

of the DNA fragment delimited by the nucleotides at positions 2193 to 2321 of SEQ ID NO: 1, thus leading to a mutated RNase E gene represented by SEQ ID NO: 3 and coding for the mutated RNase E protein RneΔ24 represented by SEQ ID NO: 4 and corresponding to SEQ ID NO: 2 wherein the sequence delimited by the aminoacids at positions 585 to 627 is deleted,

of the DNA fragment delimited by the nucleotides at positions 2193 to 2519 of SEQ ID NO: 1, thus leading to a mutated RNase E gene represented by SEQ ID NO: 5 and coding for the mutated RNase E protein RneΔ23 represented by SEQ ID NO: 6 and corresponding to SEQ ID NO: 2 wherein the sequence delimited by the aminoacids at positions 585 to 693 is deleted,

of the DNA fragment delimited by the nucleotides at positions 2247 to 2519 of SEQ ID NO: 1, thus leading to a mutated RNase E gene represented by SEQ ID NO: 7 and coding for the mutated RNase E protein RneΔ22 represented by SEQ ID NO: 8 and corresponding to SEQ ID NO: 2 wherein the sequence delimited by the aminoacids at positions 603 to 693 is deleted,

of the DNA fragment delimited by the nucleotides at positions 2247 to 2321 of SEQ ID NO: 1, thus leading to a mutated RNase E gene represented by SEQ ID NO: 9 and coding for the mutated RNase E protein RneΔ21 represented by SEQ ID NO: 4 and corresponding to SEQ ID NO: 2 wherein the sequence delimited by the aminoacids at positions 603 to 627 is deleted,

of the DNA fragment delimited by the nucleotides at positions 2346 to 2519 of SEQ ID NO: 1, thus leading to a mutated RNase E gene represented by SEQ ID NO: 11 and coding for the mutated RNase E protein RneΔ17 represented by SEQ ID NO: 12 and corresponding to SEQ ID NO: 2 wherein the sequence delimited by the aminoacids at positions 636 to 693 is deleted.

The invention relates more particularly to E. coli strains as defined above, characterized in that the inducible expression system uses RNA polymerase of the T7 bacteriophage.

The invention also concerns nucleotide sequences comprising a gene coding RNase E with a mutation such that the enzyme produced upon expression of this mutated gene exhibits reduced activity for degrading the messenger RNA (m-RNA) encoding said polypeptides or proteins, compared to bulk mRNA, said mutation not significantly affecting growth of the said E. coli strains, and wherein said mutation corresponds to the deletion :

of the DNA fragment delimited by the nucleotides at positions 2193 to 2321 of SEQ ID NO: 1, thus leading to a mutated RNase E gene represented by SEQ ID NO: 3 and coding for the mutated RNase E protein RneΔ24 represented by SEQ ID NO: 4 and corresponding to SEQ ID NO: 2 wherein the sequence delimited by the aminoacids at positions 585 to 627 is deleted,

of the DNA fragment delimited by the nucleotides at positions 2193 to 2519 of SEQ ID NO: 1, thus leading to a mutated RNase E gene represented by SEQ ID NO: 5 and coding for the mutated RNase E protein RneΔ23 represented by SEQ ID NO: 6 and corresponding to SEQ ID NO: 2 wherein the sequence delimited by the aminoacids at positions 585 to 693 is deleted,

of the DNA fragment delimited by the nucleotides at positions 2247 to 2519 of SEQ ID NO: 1, thus leading to a mutated RNase E gene represented by SEQ ID NO: 7 and coding for the mutated RNase E protein RneΔ22 represented by SEQ ID NO: 8 and corresponding to SEQ ID NO: 2 wherein the sequence delimited by the aminoacids at positions 603 to 693 is deleted,

of the DNA fragment delimited by the nucleotides at positions 2247 to 2321 of SEQ ID NO: 1, thus leading to a mutated RNase E gene represented by SEQ ID NO: 9 and coding for the mutated RNase E protein RneΔ21 represented by SEQ ID NO: 4 and corresponding to SEQ ID NO: 2 wherein the sequence delimited by the aminoacids at positions 603 to 627 is deleted,

of the DNA fragment delimited by the nucleotides at positions 2346 to 2519 of SEQ ID NO: 1, thus leading to a mutated RNase E gene represented by SEQ ID NO: 11 and coding for the mutated RNase E protein RneΔ17 represented by SEQ ID NO: 12 and corresponding to SEQ ID NO: 2 wherein the sequence delimited by the aminoacids at positions 636 to 693 is deleted.

The invention also relates to vectors, such as plasmids containing nucleotide sequences comprising a gene coding RNase E (also called rne gene) with a mutation as described above.

A general process for obtaining mutant E. coli strains as described above, and capable of being used in the context of the present invention, comprises the following steps:

preparation of a plasmid containing an rne gene comprising a mutation as described above ; the plasmid is such that its replication is thermosensitive, i.e. it cannot be maintained as a fre-replicating episome at high temperature (42° C.)

introduction of the plasmid obtained in the preceding step, into an E. coli strain comprising an inducible expression system, as well as a chromosomal mutation in the rne gene such as the so-called rne1 mutation (Ono and Kuwano, 1979) or other mutations rendering the growth of the said E. coli strain thermosensitive; this situation allows selecting for the acquisition of the desired mutation of the rne gene on the E. coli chromosome at high temperature, by forcing homologous recombination between the plasmidic and chromosomal rne sequences under conditions where neither the thermosensitive chromosomal rne allele, nor the non-replicating plasmidic rne allele, can sustain growth on its own.

The invention will be further illustrated with the following detailed description of the preparation of a mutant E. coli strain according to the invention, and of its use for producing predetermined polypeptides.

The RNA degradosome of Escherichia coli is a multienzyme complex that was discovered during efforts to purify and characterize RNase E (Carpousis et al., 1994; Carpousis et al., 1999; Miczak et al., 1996; Py et al., 1994; Py et al., 1996). The other integral components of the degradosome are enolase, an RNA helicase (RhlB) and polynucleotide phosphorylase (PNPase). RhlB is a member of the DEAD-box family of RNA helicases (Schmid and Linder, 1992). PNPase, a single-strand-specific exonuclease, is a member of the RNase PH family of 3′→5′ RNA degrading enzymes (Deutscher and Li, 2001; Symmons, 2002). Members of both families are found in a wide range of prokaryotic and eukaryotic organisms. Experiments in vitro demonstrated that RhlB in the degradosome facilitates the degradation of structured RNA by PNPase (Coburn et al., 1999; Py et al., 1996). Other ribonucleolytic complexes, e.g. the yeast exosome and mtEXO complex also have associated factors that are putative RNA helicases (de la Cruz et al., 1998; Dziembowski and Stepien, 2001; Jacobs et al., 1998; Margossian et al., 1996).

The gene encoding RNase E was identified because of its role in the maturation of E. coli 5S ribosomal RNA (Ghora and Apirion, 1978; Misra and Apirion, 1979). RNase E is a single-strand-specific endonuclease (Cormack and Mackie, 1992; Ehretsmann et al., 1992; Lin-Chao et al., 1994). Subsequent work showed that RNase E has a more general role in RNA metabolism and it is now believed to be the principal endonuclease in E. coli messenger RNA decay (Coburn and Mackie, 1999; Grunberg-Manago, 1999; Regnier and Arraiano, 2000). RNase E is a large, 1061 residue, protein (Casaregola et al., 1992). Its nucleolytic activity resides in the N-terminal half. The C-terminal half (CTH) of the protein contains a proline rich linker, an arginine rich RNA binding domain (RBD) and a region that is the scaffold for protein-protein interactions with the other components of the degradosome (Kaberdin et al., 1998; McDowall and Cohen, 1996; Taraseviciene et al., 1995; Vanzo et al., 1998). Proteins related to RNase E are found throughout the eubacterial kingdom and in some plants (Condon et al., 2001). The plant homologues are presumably in the chloroplast, which is an organelle of eubacterial origin. An RNase E-based degradosome was recently identified in Rhodobacter capsulatus (Jager et al., 2001). The complex contains two DEAD proteins and the transcription termination factor Rho, but not PNPase and enolase. E. coli encodes a paralogue of RNase E now known as RNase G (Jiang et al., 2000; Li et al., 1999; Tock et al., 2000). It has significant homology to the N-terminal catalytic domain of RNase E but is smaller because it lacks a CTH. The ‘RNase E/G’ family of proteins can thus be divided into two groups: the large RNase E-like enzymes that can form degradosomes and the small RNase G-like enzymes that apparently act alone.

In E. coli, the tight coupling between transcription and translation is important for mRNA stability. Bacteriophage T7 RNA polymerase (RNAP) elongates significantly faster than the E. coli enzyme. When lacZ mRNA is transcribed by T7 RNAP, long stretches of ribosome-free message occur. These untranslated T7-lacZ mRNAs are very unstable in uninfected cells and this effect correlates with the rate of elongation (Makarova et al., 1995). With wild-type T7 RNAP (8-fold faster than the E. coli enzyme) the β-galactosidase synthesized per message is only a few percent compared to the same transcript from E. coli RNAP. RNase E is responsible for this rapid functional inactivation (Iost and Dreyfus, 1995). In strains containing the rne131 mutation, which produces a truncated RNase E missing the CTH, there is a substantial increase in the functional stability of the T7-lacZ transcript (Lopez et al., 1999). This is a general effect since the yield of many proteins expressed by T7 RNAP can be improved in the rne131 background. Thus elements in the CTH of RNase E have an important role in the degradation of the T7 messages. Since stability can be easily measured by β-galactosidase activity, the T7-lacZ mRNA is a useful reporter to study the effect of RNase E mutations on mRNA degradation in vivo.

Another transcript that is sensitive to mutations in the CTH of RNase E is the rne message. The expression of RNase E is autoregulated by a mechanism involving the stability of its own mRNA (Jain and Belasco, 1995; Mudd and Higgins, 1993). A 361 nt 5′ untranslated region (5′ UTR) in the rne message is essential for autoregulation (Diwa et al., 2000). Catalytic activity is necessary for autoregulation but not sufficient since the CTH significantly enhances the capacity of RNase E to regulate its own synthesis (Jiang et al., 2000).

We previously made deletions within the CTH of RNase E to elucidate the protein-protein interactions in the RNA degradosome (Vanzo et al., 1998). In these constructs, the chromosomal gene was inactivated by an amber mutation and complemented with plasmids containing various mutant alleles of rne. Ow et al. (2000) exploited a similar system using a deletion of the chromosomal rne gene. Although relatively simple to construct, these systems have disadvantages including the need to work in a recombination deficient background and the concern that the dose of the complementing gene could vary due to changes in plasmid copy number. Here we report the construction and characterization of strains in which mutant alleles of rne, encoding proteins with deletions in the CTH, have been substituted directly into the chromosome, replacing the wild-type gene. The activity of RNase E in vivo was measured using the T7-lacZ reporter, RNase E expression was quantified by Western blotting, and RNA binding was examined by Northwestern blotting. In order to disrupt autoregulation and control expression in vivo, we have also constructed strains in which certain RNase E mutants were put under the control of a lac promoter. The role of the RBD and protein scaffold in controlling RNase E activity is discussed.

Results

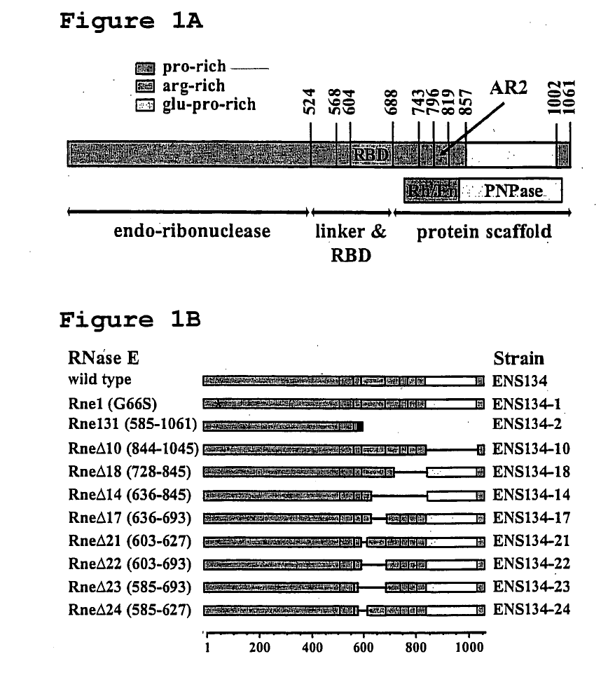

In previous work, we constructed plasmid-based alleles of rne encoding RNase E with deletions in the CTH that disrupted the linker, RBD and/or protein scaffold (Vanzo et al., 1998). With these mutants, we measured the stability of RNA1, which is a repressor of ColE1 plasmid replication (unpublished results). Several of the mutants showed significantly impaired RNA1 decay, but Northern blotting revealed that these strains were also defective in the maturation of 5S rRNA suggesting an intrinsic defect in ribonuclease activity. Since a common feature of these mutants was the deletion of the proline-rich ‘hinge’ flanking the catalytic domain, we decided to make new mutants conserving this linker region (FIG. 1A). Another observation was that several mutant proteins appeared to be strongly overexpressed. To determine if this effect was due to defective autoregulation and avoid potential variations in gene copy number, we also decided to construct the new mutants as single copy replacements of rne⁺ in the chromosome.

The ENS134 Strain and Degradation of the T7-lacZ Message

Eight new strains were constructed. FIG. 1B shows the structure of the RNase E mutant proteins studied here and indicates the name of the ENS134 derivatives encoding these proteins. Rne1 and Rne131 are controls that have been characterized previously (see FIG. legend). In all of the new strains growth is normal in rich media at 30, 37 and 420° C.; the maturation of 5S ribosomal RNA is not perturbed; and the mutant proteins are stable as judged by experiments in which protein synthesis was inhibited then RNase E levels were followed by Western blotting (data not shown).

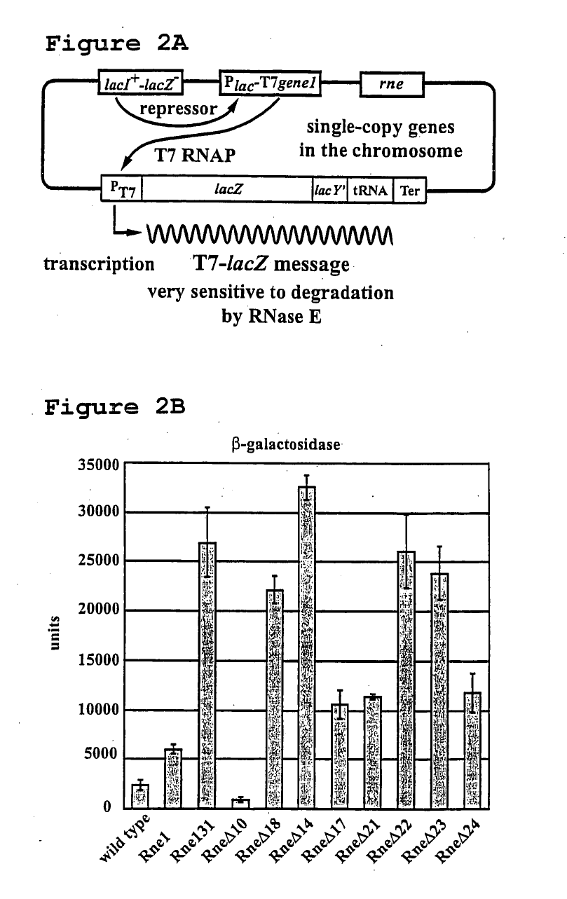

The ENS134 strain encodes a hybrid lacZ mRNA is expressed by the bacteriophageT7 RNAP (FIG. 2A). Although the 5′ leader and more than 3000 nt of coding sequence are identical to authentic lacZ mRNA, we will refer to this message as T7-lacZ to distinguish it from RNA transcribed by E. coli RNAP. FIG. 2B shows the level of expression of the T7-lacZ gene as measured by β-galactosidase synthesis. Since expression from a single-copy T7-lacZ gene is not toxic, the strains were cultured continuously in the presence of IPTG and activity was measured during early logarithmic growth. Rne1 and Rne131 have higher levels of β-galactosidase in agreement with previous work showing that this is due to the stabilization of the T7-lacZ message (Iost and Dreyfus, 1995; Lopez et al., 1999). This experiment was performed at 30° C., which is the permissive temperature for growth with the rne1 allele. Even under these conditions, the Rne1 protein is defective as evidenced by the approximately 2-fold increase in β-galactosidase activity. With the exception of RneΔ10, all of the mutant proteins exhibited increased β-galactosidase expression ranging from about 5-fold (RneΔ17, Δ21 and Δ24) to 13-fold (RneΔ14). The β-galactosidase activity for several of the proteins (RneΔ18, Δ14, Δ22 and Δ23) is comparable to that of Rne131 lacking the entire CTH. The deletion of the RBD (RneΔ22 and Δ23) or the region that binds RhlB and enolase (RneΔ18) affects β-galactosidase levels equivalently (about 10-fold) whereas the deletion of both regions (RneΔ14) is comparable to Rne131 . Previous experiments demonstrated that the deletion in RneΔ18 disrupts the interaction with RhlB and enolase, but not PNPase (Vanzo et al., 1998). Thus both the RBD and the region of the scaffold interacting with RhlB and enolase are necessary for efficient degradation of the T7-lacZ mRNA.

The result with RneΔ10 was unexpected. We reproducibly observe a reduction in β-galactosidase (40% of wild-type). This strain expresses the RneΔ10 protein, which lacks the acidic C-terminal region of RNase E. Previous experiments demonstrated that this deletion specifically disrupts the interaction between RNase E and PNPase (Vanzo et al., 1998). Our result suggest that RneΔ10 is more active in the degradation of the T7-lacZ mRNA than wild type RNase E.

Autoregulation of Mutant RNase E Expression

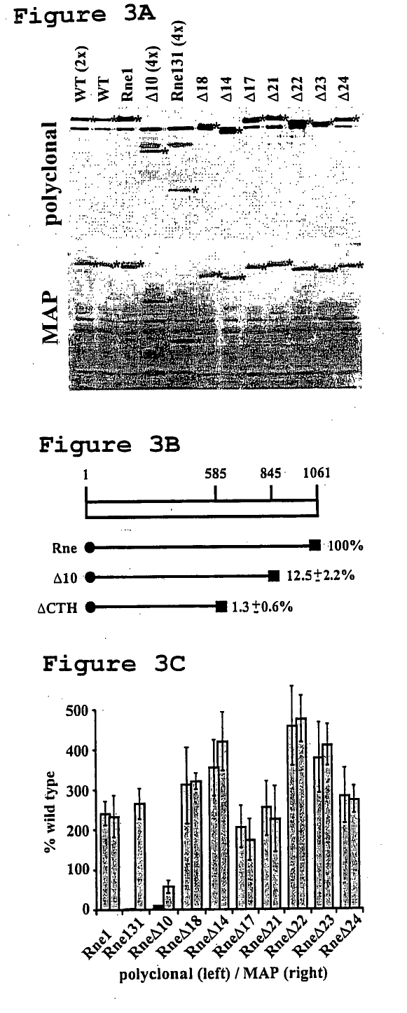

The expression of RNase E was quantified by Western blotting using two antibodies. In FIG. 3A, a polyclonal rabbit antibody raised against the entire Rne protein (top panel) or a MAP antibody (bottom panel) against the N-terminal 20 residues of RNase E was used. The blots were developed using a fluorescent reaction (ECF) and quantified with a fluorimager (FIG. legend and Experimental procedures). Note that with the polyclonal antibody the signal from RneΔ10 and Rne131 is very weak compared to wild-type RNase E and the other mutants even though more protein was loaded and the fluorescent signal with the other proteins is saturated in this image. Since Rne131 and RneΔ10 are both missing the C-terminal region whereas the other mutants have this domain (FIG. 1B), we tested the possibility that the polyclonal antibody reacts preferentially with the last 200 amino acids of RNase E. Three N-terminal His-tagged forms of RNase E were constructed and purified (FIG. 3B). Δ10 is equivalent to the protein discussed above except for the tag (black circle, left). ΔCTH is similar to Rne131 except that like RneΔ10 it contains the C-terminal 16 amino acids of RNase E (black square, right). The reactivity of equivalent weights of each protein was quantified by ECF Western blotting (data not shown). In FIG. 3B, the fluorescence per weight for each protein, normalized to full-length RNase E, is shown to the right. The signal with RneΔ10 is only 12.5% of wild type thus confirming that the polyclonal antibodies react preferentially with the C-terminal domain of RNase E. Furthermore, the signal from the RneΔCTH mutant is only 1.3% showing that most of the epitopes detected by our polyclonal antibody are in the CTH of RNase E thus explaining the weak signals from Rne131 and RneΔ10.

We have quantified the level of expression of the Rne mutant proteins (FIG. 3C, see legend and Experimental procedures for details). These results are normalized to the level of wild type RNase E. Comparison of the data from the polyclonal and MAP antibodies show that they are equivalent except for Rne131 and RneΔ10. If we correct the results for Rne131 and RneΔ10 with the polyclonal antibodies using the values in FIG. 3B, we get very good agreement with the data from the MAP antibodies (not shown). The proteins levels vary from 65% (RneΔ10) to 490% (RneΔ22), which is a 7.5-fold difference. Disruption of the RBD and/or the region that binds RhlB and enolase leads to overexpression whereas the RneΔ10 mutant is underexpressed. The synthesis of the mutant proteins is similar to β-galactosidase expression (compare FIG. 3C to FIG. 2B) showing that there is an anti-correlation between RNase E levels and the degradation of the T7-lacZ mRNA.

RNA Binding by Mutant RNase E Proteins

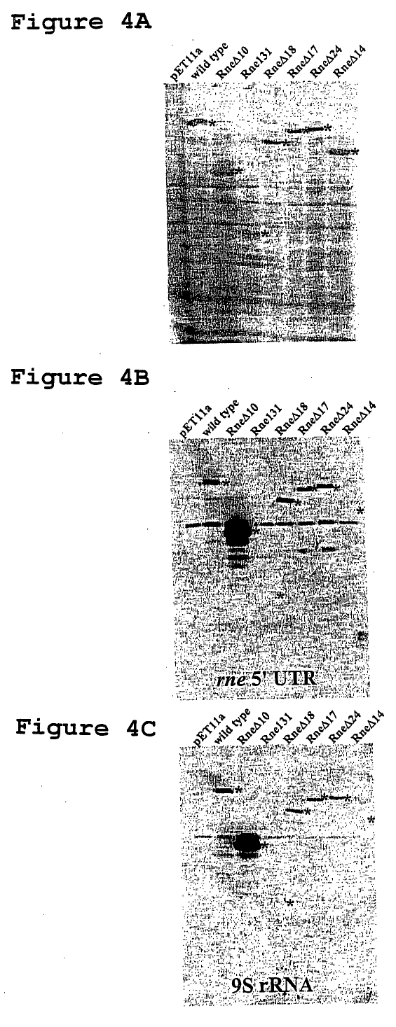

RNA binding by RNase E was originally demonstrated using Northwestern blotting (Cormack et al., 1993). This technique was used in subsequent studies to more precisely map the RBD (McDowall and Cohen, 1996; Taraseviciene et al., 1995). We thus decided to analyze our mutant proteins using the same procedure. The proteins were overexpressed, separated by SDS-PAGE, and the amount of each mutant protein was estimated by fluorimaging. FIG. 4A shows a SYPRO Orange stained gel in which the loading of total protein was varied to give comparable amounts of each mutant protein. In parallel, blots were probed with radioactive RNA and analyzed by phosphorimaging. In FIG. 4B, one blot was probed with the 5′ UTR of the rne mRNA (upper panel) and the other with 9S rRNA (lower panel). The results with the two substrates are equivalent. As expected, wild type RNase E, RneΔ10 and RneΔ18, which contain the RBD, bind RNA whereas Rne131 and RneΔ14, which have deletions of the RBD, do not. Surprisingly, the other mutants on the blot, RneΔ17 and RneΔ24, bind RNA with a signal comparable to wild-type RNase E. Note that RneΔ14 (636-845) does not bind RNA whereas RneΔ17 (636-693) does. This shows that an element between 693 and 845, most likely AR2 (see FIG. 1A), can bind RNA independently of the RBD. In other experiments, we observed that RneΔ21 (603-627), RneΔ22 (603-693) and RneΔ23 (585-693) also bind RNA (data not shown).

In Northwestern blotting, RneΔ10 reproducibly has a stronger signal (5-10-fold more radioactivity by weight) than wild type or the other mutants that bind RNA (FIG. 4B). This signal, which results from the retention of significantly more RNA after washing the blot, suggests that the capacity and/or affinity of RneΔ10 for binding RNA is higher than wild type. This increased RNA binding correlates with the higher activity of RneΔ10 in vivo as judged by T7-lacZ activity and the autoregulation of RNase E expression. A model that could explain the influence of the acidic C-terminal domain on RNA binding is presented in the Discussion.

RNase E Autoregulation Compensates for Defective Degradation Activity

The effect of the mutations on T7-lacZ mRNA stability might be underestimated as a consequence of autoregulation. For instance, with the Rne131 mutant protein we see 11-fold more β-galactosidase activity (FIG. 2B). However, considering that Rne131 is 3-fold overexpressed (FIG. 3C), the defect appears to be more serious. Table 1 shows an analysis of the activity in vivo of the mutant proteins. Note that the activity of mutants such as RneΔ18, RneΔ14, RneΔ22, and RneΔ23 is at least as defective as rne131 in both the autoregulation of RNase E synthesis (relative RNase E levels) and the degradation of the T7-lacZ mRNA (specific degradation efficiency). This analysis rests on the assumption that alterations in the concentration of the mutant proteins by autoregulation changes the level of mRNA degrading activity. To test this, we constructed new strains in the ENS134 background in which the promoter and the 5′ UTR of rne were replaced with the promoter and leader of the lacZ gene. The construction and characterization of related strains with rne under P_{lac} control has been recently described (Sousa et al., 2001). Table 2 shows a measurement of the degradation of the T7-lacZ mRNA when rne⁺, RneΔ10 and rne131 are autoregulated (first column, Pᵣₙₑ) or expressed equivalently (second column, P_{lac}). By Western blotting, the expression of RNase E and the mutant proteins from P_{lac} was about 2-fold higher than the wild type protein with normal autoregulation (data not shown). The lower β-galactosidase activity measured with wild type rne under P_{lac} control (770 vs. 2370 units) could be due to destabilization of the T7-lacZ mRNA by the 2-fold higher expression of RNase E. Note that under conditions of equivalent expression (P_{lac}), the ratio of β-galactosidase activity in wild type to mutant is 4.0 and 0.028 for RneΔ10 and rne131 respectively. This is in very good agreement with the specific degradation efficiency of 4.3 and 0.034 in Table 1 confirming the validity of the calculation. The comparison of the results in the first (Pᵣₙₑ) and second (P_{lac}) columns in Table 2 shows that autoregulation compensates, at least partially, for defective degradation of the T7-lacZ mRNA. Although it would have been interesting to perform this analysis over a range of IPTG concentrations, the growth defects with the P_{lac}-rne131 strain described in the next section precluded further analysis.

Aberrant Growth of the P_{lac}-rne¹³¹ Strain

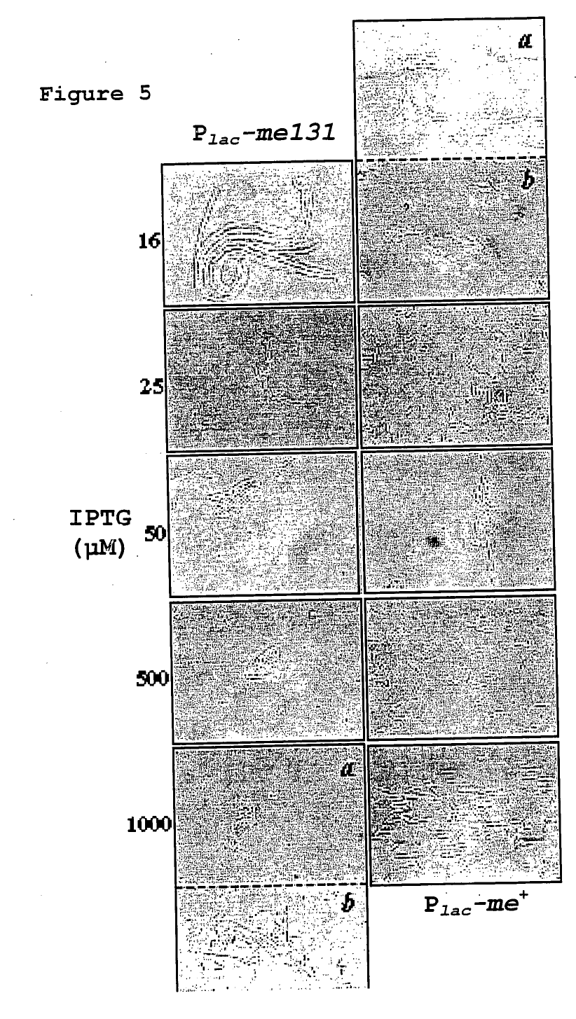

The strains in which rne is under the control of P_{lac} require IPTG for growth. On solid media there is no visible colony formation in the absence of IPTG. Upon examining the growth of the P_{lac}-rne131 strain at different IPTG concentrations, we noticed significant filamentation at both low and high inducer concentrations. FIG. 5 shows representative images of microcolony formation of the P_{lac}-rne131 strain over a range of IPTG concentrations. The P_{lac}-rne⁺ strain is shown for comparison. The morphology at 16 μM IPTG is striking. The colony appears to be composed a few very long filaments. At 25 and 50 μM inducer long filaments predominate although some normal cells appear at the higher concentration. With the wild-type RNase E control, at 16 μM IPTG we observed two different types of colonies: some filaments (a) vs. normal (b). Above this concentration, cell morphology is normal in the P_{lac}-rne⁺ strain. At 500 uM IPTG the morphology of P_{lac}-rne131 is normal whereas at 1000 uM we observed two different types of colonies: normal (a) vs. long filaments (b). In control experiments, the Pᵣₙₑ-rne131 strain, which has autoregulated expression, showed normal cell morphology throughout the range of IPTG concentrations tested here (data not shown). Staining of the filamented cells with DAPI showed that DNA replication and partition of the nucleoid appears normal (data not shown). However, the cells fail to form septums and divide. These results show that growth of the P_{lac}-rne131 cells depends on the dose of IPTG whereas the P_{lac}-rne⁺ strain is normal except at the lowest concentration of inducer. The P_{lac}-rne131 strain has a clearly discernable defect in cell division, at levels of expression where wild type is normal, adding further support to the idea that autoregulation has an important role in compensating for defective RNase E activity.

Fitness of Strains with Mutant RNase E Proteins

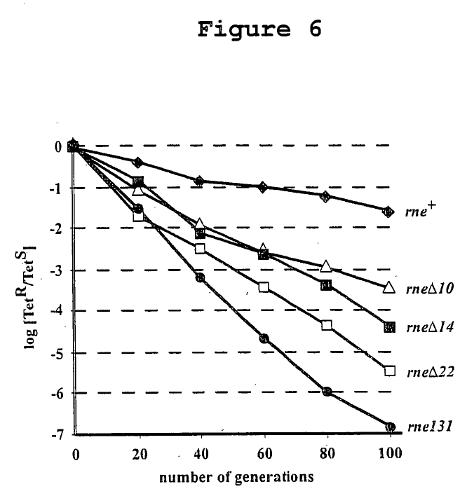

The strains where the expression of the mutant proteins is autogenously controlled have a discernable phenotype with respect to the stability of the T7-lacZ mRNA and RNase E expression. However, the rate of logarithmic growth in liquid media is unaffected although small differences might not be detected by this analysis. A sensitive test of a mutant strain is its ‘fitness’ in competition with an isogenic ‘wild type’ control. FIG. 6 shows the results of such an experiment. Wild type (rne⁺) is a derivative of MC1061 containing a Tn10 transposon linked to the rne locus. The mutants are genetically identical except for the alteration in the rne gene. The Tn10 containing strains can be distinguished from the parent by their resistance to tetracycline. MC1061 (Tet^{S}) and each of the Tn10 derivatives (Tet^{R}) were mixed together in equal proportions then grown for 100 generations. Each 20 generations, cells were plated to measure the proportion that was tetracycline resistant. With the rne⁺ control, there is a small loss, less than 100-fold per 100 generations, due to the Tn10 insertion. All of the mutants are lost significantly faster. After 100 generations, rneΔ10 is approximately 100-fold lower than rne⁺; rne Δ14, 1000-fold; rneΔ22, 10,000-fold; and rne131 , 100,000-fold. FIG. 6 shows the results of a single experiment. This work was repeated once in an experiment that ran for 60 generations. The results were comparable to experiment shown in FIG. 6. The mildest defects involve the deletion of the acidic C-terminal domain (RneΔ10 ) and the region that binds RhlB and enolase (rneΔ14). Deletion of the RBD (rneΔ22) is more severe whereas removal of the entire CTH (rne131 ) has the largest effect. This experiment demonstrates that, even with autoregulation, mutations in the rne locus that disrupt the non-catalytic part of RNase E significantly diminish the fitness of these strains.

Discussion

The evidence that RNase E can form a complex with other enzymes involved in the RNA degradation is overwhelming. In addition to the work cited in the Introduction, a ‘minimal’ RNA degradosome has been reconstituted from purified RNase E, PNPase and RhlB (Coburn et al., 1999) and a recent study by immunoelectron microscopy has shown that RhlB co-localizes with RNase E in a CTH-dependent manner in vivo (Liou et al., 2001). PNPase is distributed throughout the cell (Py et al., 1994), but it is in large excess relative to RhlB and RNase E (Liou et al., 2001). We have previously argued, based on heterogeneity in sedimentation, that the interaction of PNPase with RNase E might be dynamic (Carpousis et al., 1994). Thus, in the cell, PNPase could partition between free and degradosome-bound fractions. The endonuclease activity of RNase E is necessary for viability, but strains expressing mutant proteins missing the non-catalytic part of RNase E can grow.

The rne131 mutation was isolated in a screen for extragenic suppressors of a temperature-sensitive mukB allele (Kido et al., 1996). The suppression resulted from a small (2-fold) overexpression of the mutant MukB protein. Several other RNase E mutants were obtained in this screen. All of them result in a truncated protein. The rne131 mutation was extensively characterized in a subsequent study which showed that the maturation of 5S ribosomal RNA was normal whereas there was a small (2-fold) slowing in the chemical and functional decay of bulk mRNA (Lopez et al., 1999). This study also showed that certain messages such as the T7-lacZ mRNA and the endogenous thrS mRNA were preferentially stabilized compared to bulk mRNA. Here we have exploited the sensitivity of the T7-lacZ mRNA to map the regions in the non-catalytic part of RNase E that affect mRNA degradation. Our work shows that deletions in different regions of the non-catalytic part of RNase E have opposite effects on the degradation of the T7-lacZ mRNA. Removal of the RBD and/or the region that binds RhlB and enolase reduces mRNA-degrading activity, whereas deletion of the acidic C-terminal domain increases it. Expression of the mutant RNase E proteins anti-correlates with mRNA-degrading activity. The hypoactive mutants are overexpressed whereas the hyperactive RneΔ10 mutant is underexpressed.

Ow et al. (2000) were the first to publish an in vivo analysis of the function of mutants that disrupted the RBD or protein scaffold. By examining the decay of a battery of endogenous mRNAs, it was concluded that the deletion of the entire protein scaffold did not affect mRNA degradation whereas the deletion of the RBD had only a small effect and the deletion of both significantly slowed mRNA decay. These results were interpreted as evidence that an RNase E-based degradosome is not necessary for normal mRNA degradation. Our results support the conclusion that the RBD has a role in mRNA decay but we also observe significant effects on the degradation of the T7-lacZ mRNA with deletions in the protein scaffold. One possible explanation of this difference is that Ow et al. (2000) deleted the entire protein scaffold whereas we deleted subregions that have opposite effects on mRNA degradation (see above). Alternatively, it is conceivable that there is a genuine difference between the pathway by which normally translated vs. ribosome-free mRNA is degraded. Indeed, the striking parallel between the effect of the deletions on the degradation of the T7-lacZ mRNA and the autoregulation of RNase E synthesis suggests that the mechanism of degradation of ribosome-free mRNA and rne autoregulation are related. The T7-lacZ message is ribosome-free in the sense that the T7 RNAP largely outpaces the ribosome. The 361 nt rne 5′ leader is an untranslated target. It is noteworthy that certain other messages have been shown to be preferentially stabilized by the rne131 allele, e.g. the thrS mRNA encoding threonyl-tRNA synthetase (Lopez et al., 1999). ThrS autogenously regulates its own expression by binding to an ‘operator’ in the 163 nt 5′ leader of its mRNA and inhibiting translation. Recent work shows that in rne⁺ cells the repressed thrS mRNA is rapidly degraded whereas with rne131 it accumulates significantly (Nogueira et al., 2001). Thus the results with T7-lacZ, rne and thrS mRNA all suggest that elements in the non-catalytic part of RNase E facilitate the preferential degradation of ribosome-free mRNA. We speculate that this process might represent a type of ‘mRNA surveillance’ in which the uncoupling of translation from transcription triggers decay.

All of the RNase E mutants studied here bind RNA except Rne131 and RneΔ14. This activity was initially localized to the CTH (Cormack et al., 1993) then more precisely mapped to the RBD (McDowall and Cohen, 1996; Taraseviciene et al., 1995). Our results suggest that a second arginine rich region (AR2, residues 796-819, 50% arginine) can also bind RNA. Analysis of the published work (McDowall and Cohen, 1996; Taraseviciene et al., 1995) shows that the constructs employed in those studies would not have permitted the detection of an independent RNA binding site at AR2. An important conclusion of our work is that there is no simple correlation between RNA binding in vitro and mRNA-degrading activity in vivo. Mutants such as RneΔ18, with an intact RBD and RNA binding activity, exhibit significant defects in T7-lacZ mRNA degradation and rne autoregulation. Our results show that RNA binding is more complex then anticipated: the RNA and protein binding sites overlap and they cannot be separated by simple deletions. Considering the effect of deletions located between residues 585 to 845 on the degradation of the T7-lacZ mRNA, this entire region appears to comprise either a single functional domain or perhaps two domains that cooperate in the same reaction.

The observation that the deletion of the last 200 residues of RNase E (RneΔ10) leads to destabilization of the T7-lacZ message and reduces expression of the mutant protein is novel. These results suggest that RneΔ10 in vivo is more active than wild type. Recent work (A.L., unpublished) shows that the RneΔ10 mutation also destabilizes the T7-lacZ mRNA in a pnp⁻ strain. The pnp⁻ allele, the same that was used by Lopez et al. (1999), abolishes production of PNPase, which is not detectable by Western blotting. This shows that, even though the region deleted in RneΔ10 is involved in an interaction with PNPase, the phenotype of rneΔ10 does not involve PNPase per se. A plausible explanation is that the acidic C-terminal domain is a negative effector of RNase E activity. Since the removal of the acidic region stimulates RNA binding (FIG. 4), this domain appears to be an inhibitor of the basic RBD and/or AR2 regions. It should be interesting to explore this interaction in future work.

The growth defects in the P_{lac}-rne131 strain at low and high IPTG concentrations is evidence that autoregulation is important in compensating for the defective activity (FIG. 5). As noted recently, using P_{lac} control, wild type RNase E expression can be substantially reduced below normal levels (Jain et al., 2002; Sousa et al., 2001). This is also observed with our P_{lac}-rne⁺ construct since growth is normal except at the lowest concentration (16 μM). However, the growth of the P_{lac}-rne131 construct is already defective at 50 μM IPTG and grossly aberrant at lower concentrations. These results demonstrate for the first time the role of autoregulation in compensating for defective activity and the importance of the non-catalytic part of RNase E for normal function in vivo. Even with autoregulation, the mutant strains are disadvantaged when grown in competition with wild type (FIG. 6). After 100 generations of growth, there is only 1 cell harboring the rne131 mutant for every 100,000 that are rne⁺. Even with the least impaired mutant (rneΔ10), only about 1% remains after 100 generations. In this protocol, since the cells reach stationary phase between dilutions, there is competition at each stage of growth: i.e. the transition from stationary phase, logarithmic growth, the transition to stationary phase and survival thereafter. Although we do not know the specific defect that causes the growth phenotype, as already discussed, rne131 slows bulk mRNA decay and preferentially stabilizes certain messages. Thus it seems reasonable to believe that the disadvantage involves a problem with mRNA degradation.

Experimental Procedures

Standard Techniques, Strains and Plasmids

General techniques in genetics and molecular biology have been described (Miller, 1972; Sambrook and Russell, 2001). The strains and plasmids used here are listed in Tables 3 and 4.

The ENS134 derivatives were constructed as follows: rneΔ10, rneΔ14 and rneΔ18 were previously generated in the pAM-rne plasmid by inverse PCR as described (Vanzo et al., 1998). The PstI fragments containing these mutant rne alleles were cloned into the NsiI site of pLN135.1 (Cornet et al., 1996) to generate pLN-rneΔ10, pLN-rneΔ14 and pLN-rneΔ18. The others deletions were generated by inverse PCR using the pLN-rne plasmid (Tbl. 4). The pLN-Δrne plasmids were transformed into AC23 (Vanzo et al., 1998). Using a previously described protocol (Cornet et al., 1996), the endogenous temperature-sensitive rne1 allele was replaced by the mutant alleles. Briefly, the protocol relies on the following elements. The target strain is resistant to streptomycin and harbors a temperature-sensitive allele (rne1 ) that does not permit growth at 42° C. The plasmid contains a temperature-sensitive origin that does not permit replication at 42° C., a cat gene conferring resistance to chloramphenicol and an rpsL⁺ gene that renders the integrants sensitive to streptomycin. After transformation and selection for chloramphenicol resistance at 30° C., colonies are streaked onto plates with chloramphenicol at 420° C. to select integrants, which grow since the rneΔ alleles are not temperature-sensitive. Excision relies on the selection of streptomycin resistant cells at 42° C., which were then tested for sensitivity to chloramphenicol. The replacements were verified by PCR and Western blotting. P1 transduction was used to move the mutant rne alleles from the AC background to ENS 134-1 (Lopez et al., 1999). The transductants were selected at 42° C. and the replacement of the endogenous temperature-sensitive rne1 allele was verified by PCR and Western blotting. Throughout the experiments in this work, two independently derived strains for each deletion were analyzed. In every case, both strains gave the same results. For simplicity, we only show the results for one set of strains.

The ENS134(P_{lac}) derivatives were constructed as follows. Replacement of the promoter and 5′ UTR of the rne gene by the corresponding region of the lac operon using a pKO3 derivative has already been described (Sousa et al., 2001). To obtain ENS134(P_{lac})-2 and ENS134(P_{lac})-10, the pKO3 derivative was transformed into ENS134-2 and ENS134-10 respectively to replace the rne 5′ control region. The same method was used to obtain ENS134(P_{lac}), except that the rne 5′ control region was first replaced in a derivative of MG1655 carrying a Tn10 transposon linked to the rne gene (zce-726::Tn10, Mudd et al., 1990). The P_{lac}-rne⁺ gene was then transduced by P1 into ENS134, by selecting for tetracycline resistance and IPTG-dependent growth.

Antibodies and Western Blotting

Rabbit polyclonal antibodies, raised against full-length RNase E, were described previously (Vanzo et al., 1998). MAP antibodies against RNase E, supplied by S. Kushner, were used in pilot experiments. We subsequently used an antibody that we produced ourselves. Briefly, a peptide corresponding to the N-terminal 20 amino acids of RNase E was synthesized using a MAP-8 matrix (Alta Bioscience, UK). The crude product was suspended in 2 ml of 10 mM Tris, pH 7.5, 1 mM EDTA, 50 mM NaCl, 0.1% SDS then sonicated and dialyzed against three changes of the same buffer. A portion of this suspension was used without further purification to raise antisera in rabbits (Eurogentec, Belgium).

SDS PAGE (7.5%) was used to separate proteins from crude extracts, which were prepared by boiling cells in SDS sample buffer (Carpousis et al. 1994). The gel was blotted to a PVDF membrane (Amersham) as described (Vanzo et al., 1998,). The blot was blocked for 2 hours in Tris buffered saline (TBS) containing 5% non fat milk, then incubated either 1 h in TBS containing 1% nonfat milk and the polyclonal antisera (1/10000 dilution) or 2 h in this buffer with the MAP antisera (1/20 dilution). The membrane was washed three times for 10 min with TBS containing 1% nonfat milk then incubated with an alkaline phosphatase coupled secondary antibody (Sigma, 1/5000 dilution) for 1 h. The membrane, washed twice for 30 min in TBS containing 1% nonfat milk and once for 30 min in TBS, was treated with ECF substrate (Amersham) and analyzed with a fluorimager (Molecular Dynamics). For quantification, each blot contained at least 2 different amounts of protein from the wild-type rne strain. During the ECF reaction the blot was scanned several times to obtain a series of images with different intensities of fluorescence. To quantify the levels of the wild type and mutant proteins, images in which the signals from the mutant protein and at least one of the wild type controls were not saturated were analyzed digitally (ImageQuant) in a procedure involving the subtraction of the background from regions just above and below the band.

Northwestern Blotting

BL21(DE3) strains containing pET11a, pET11-rne, or the pET11-rneΔ plasmids were plated on LA ampicillin (50 μg/ml) and grown overnight. A single fresh colony was inoculated in LB ampicillin (50 μg/ml) and grown at 37° C. for 4 hours (OD₆₀₀ less than 0.3). The culture were then diluted in the same medium to an OD₆₀₀ of 0.1 and grown to an OD₆₀₀ of 0.3. Expression was induced with 1 mM IPTG and the cultures were incubated for 2 h at 30° C. Total protein was prepared as described above for Western blotting. In a preliminary step, the extracts were separated by SDS-PAGE, the gel was stained with Sypro Orange (Interchim, Steinberg et al., 1996) and the amount of RNase E or mutant protein was estimated by fluorimaging. Based on this determination, the amount of total protein for Northwestern blotting was varied to give comparable amounts of RNase E and the mutant proteins.

Two probes, 9S rRNA and the 5′ UTR of the rne mRNA, were synthesized as follows. Templates were generated by PCR as described (Carpousis et al., 1994; Ehretsmann et al., 1992) using the plasmids pLN-rne (5′UTR-rne) or pKK238.8 (9S rRNA). The products, 415 bp rne-5′ UTR template and 268 bp 9S rRNA template, were purified on 3% small fragment agarose (Appligene) and extracted from the gel with a Qiaquick kit (Quiagen). The templates were transcribed in vitro using a T7 RNA polymerase kit (Promega) and ³³P-α-UTP (Amersham) then desalted on Sephadex G25. The 9S RNA was described previously (Carpousis et al., 1994). The rne-5′ UTR probe starts 4 nt before the normal transcription start of the rne mRNA (GGCCGUUUC, the underlined sequence is the normal 5′ end) and ends 47 nt after the AUG translation initiation codon. Blotting and probing were performed as described (Cormack et al., 1993), except that the membranes were incubated with the RNA probe at room temperature for 2 hours and washed (30 min) in TEN buffer with 0.02% Tween 20 and 50 mM NaCl then washed in the same buffer containing 200 mM NaCl and finally 500 mM NaCl. The radioactive RNA was visualized with a Phosphorimager (Fuji).

TABLE 1
Activity of the mutant proteins in the degradation of
the T7-lacZ mRNA and the regulation of rne expression^{a}

| | relative | relative | specific |
|---|---|---|---|
| | degradation | RNase E | degradation |
| allele | efficiency | level | efficiency |
| rne1 | 0.40 | 2.3 | 0.17 |
| rne131 | 0.090 | 2.7 | 0.034 |
| rneΔ10 | 2.5 | 0.58 | 4.3 |
| rneΔ18 | 0.11 | 3.2 | 0.034 |
| rneΔ14 | 0.074 | 4.2 | 0.018 |
| rneΔ17 | 0.23 | 1.7 | 0.13 |
| rneΔ21 | 0.21 | 2.2 | 0.094 |
| rneΔ22 | 0.092 | 4.8 | 0.019 |
| rneΔ23 | 0.10 | 4.2 | 0.025 |
| rneΔ24 | 0.20 | 2.7 | 0.075 |
| ^{a}The data from FIGS. 2B and 3C (MAP antibody) was analyzed as follows. Relative degradation efficiency of the T7-lacZ mRNA is the β-galactosidase activity of wild type divided by the mutant, e.g. 1.0 = wild type, and 0.2 = 5-fold more β-galactosidase. Relative RNase E level is the amount of mutant RNase E divided by wild type, e.g. 1.0 = wild type, and 5.0 = 5-fold more RNase E. Specific degradation efficiency is obtained by dividing the | | | |
| #value in the first column by the value in the second column, e.g. 1.0 = wild type and 0.2 = 5-fold more β-galactosidase for the same amount of RNase E. | | | |

TABLE 2
Degradation of the T7-lacZ mRNA with rneΔ10 and rne131
under P_{lac} control

| | Pᵣₙₑ | P_{lac} |
|---|---|---|
| rne⁺/rneΔ10 | 2.5 | 4.0 |
| rne⁺/rne131 | 0.089 | 0.028 |
| ^{a}The strains contain rne⁺, rneΔ10 and rne131 under the control of their own promoter and 5′ UTR (Pᵣₙₑ), or the lactose promoter and 5′ leader (P_{lac}). Degradation of the T7-lacZ mRNA is expressed as the β-galactosidase activity of wild type divided by the mutant (compare with Tbl. 1, first column). β-galactosidase activity was measured during growth at 30° C. with 100 μM IPTG. The activity of | | |
| #rne⁺was 2370 units under Pᵣₙₑ control and 770 units under P_{lac} control (see text). | | |

TABLE 3
Strains

| strain | characteristic | reference |
|---|---|---|
| AC21 | MC1061, zce-726::Tn10 | Carpousis et |
| | | al., 1994 |
| AC23 | MC1061, zce-726::Tn10, rne1(ams) | Vanzo et al., |
| | | 1998 |
| AC24 | AC23, rneΔ10 (aaΔ 844-1045)^{a} | this work |
| AC26 | AC23, rneΔ18 (aaΔ 728-845) | this work |
| AC27 | AC23, rne131 (made by P1 | this work |
| | transduction) | |
| AC28 | AC23, rneΔ14 (aaΔ 636-845) | this work |
| AC29 | AC23, rneΔ17 (aaΔ 636-693) | this work |
| AC31 | AC23, rneΔ21 (aaΔ 603-627) | this work |
| AC32 | AC23, rneΔ22 (aaΔ 603-693) | this work |
| AC33 | AC23, rneΔ23 (aaΔ 585-693) | this work |
| AC34 | AC23, rneΔ24 (aaΔ 585-627) | this work |
| ENS134 | BL21 (DE3), P_{lac}-T7 RNA polymerase, | Lopez et al., |
| | P_{T7}-lacZ | 1994 |
| ENS134-1 | ENS134, zce-726::Tn10, rne1(ams) | Iost and |
| | | Dreyfus, 1995 |
| ENS134-2 | ENS134, rne 131 | Lopez et al., |
| | | 1999 |
| ENS134-10 | ENS134, zce-726::Tn10, rneΔ10 | this work |
| | (aaΔ 844-1045) | |
| ENS134-18 | ENS134, zce-726::Tn10, rneΔ18 | this work |
| | (aaΔ 728-845) | |
| ENS134-14 | ENS134, zce-726::Tn10, rneΔ14 | this work |
| | (aaΔ 636-845) | |
| ENS134-17 | ENS134, zce-726::Tn10, rneΔ17 | this work |
| | (aaΔ 636-693) | |
| ENS134-21 | ENS134, zce-726::Tn10, rneΔ21 | this work |
| | (aaΔ 603-627) | |
| ENS134-22 | ENS134, zce-726::Tn10, rneΔ22 | this work |
| | (aaΔ 603-693) | |
| ENS134-23 | ENS134, zce-726::Tn10, rneΔ23 | this work |
| | (aaΔ 585-693) | |
| ENS134-24 | ENS134, zce-726::Tn10, rneΔ24 | this work |
| | (aaΔ 585-627) | |
| ENS134(P_{lac}- | ENS134, zce-726::Tn10, P_{lac}-rne | this work |
| rne) | | |
| ENS134 (P_{lac}- | ENS134, P_{lac}-rne131 | this work |
| rne)-2 | | |
| ENS134 (P_{lac}- | ENS134, zce-726::Tn10, P_{lac}-rneΔ10 | this work |
| rne)-10 | | |

^{a}This notation (aaΔ 844-1045) refers to the amino acids deleted in the protein expressed from the mutant rne allele. The alleles rneΔ17, rneΔ21, rneΔ22, rneΔ23 and rneΔ24 were generated for the first time in this study (Experimental procedures). The alleles rneΔ10, rneΔ18, rneΔ14 and rne131 were described previously (Kido et al., 1996; Lopez et al., 1999; Vanzo et al., 1998).

TABLE 4
Plasmids

| plasmid | characteristic | reference |
|---|---|---|
| pLN135.1 | Low copy number, temperature- | Cornet et |
| | sensitive replicon, multiple | al., 1996 |
| | cloning site, cat gene and rpsL⁺ | |
| | (Sm^{s}) | |
| pLN-rne | 6kb PstI-PstI genomic fragment | this work |
| | containing the entire rne gene | |
| | cloned into the NsiI site of | |
| | pLN135.1 | |
| pLN-rneΔ10 | derivative of pLN-rne (aaΔ 844-1045)^{a} | this work |
| pLN-rneΔ18 | derivative of pLN-rne (aaΔ 728-845) | this work |
| pLN-rneΔ14 | derivative of pLN-rne (aaΔ 636-845) | this work |
| pLN-rneΔ17 | derivative of pLN-rne (aaΔ 636-693) | this work |
| pLN-rneΔ21 | derivative of pLN-rne (aaΔ 603-627) | this work |
| pLN-rneΔ22 | derivative of pLN-rne (aaΔ 603-693) | this work |
| pLN-rneΔ23 | derivative of pLN-rne (aaΔ 585-693) | this work |
| pLN-rneΔ24 | derivative of pLN-rne (aaΔ 585-627) | this work |
| pET11a | vector for protein expression in | Studier et |
| | E. col. | al., 1990 |
| pET11a-rne | contains complete rne coding | Vanzo et |
| | sequence and transcription | al., 1998 |
| | termination site | |
| pet11a- | derivative of pET11a-rne (aaΔ 844-1045) | this work |
| rneΔ10 | | |
| pet11a- | derivative of pET11a-rne (aaΔ 728-845) | this work |
| rneΔ18 | | |
| pet11a- | derivative of pET11a-rne (aaΔ 636-845) | this work |
| rneΔ14 | | |
| pet11a- | derivative of pET11a-rne (aaΔ 636-693) | this work |
| rneΔ17 | | |
| pet11a- | derivative of pET11a-rne (aaΔ 603-693) | this work |
| rneΔ22 | | |
| pet11a- | derivative of pET11a-rne (aaΔ 585-693) | this work |
| rneΔ23 | | |
| pet11a- | derivative of pET11a-rne (aaΔ 585-627) | this work |
| rneΔ24 | | |
| pet11a- | derivative of pET11a-rne (aaΔ | this work |
| rneΔ26 | Δ585-845) | |
| pet11a- | derivative of pET11a-rne (aaΔ 585-1045) | this work |
| rneΔCTH | | |
| pET15b | derivative of pET11a with N- | Novagen |
| | terminal histidine tag | |
| pET15b-rne | rne transplanted from pET11a-rne | this work |
| | to pET15b | |
| pET15b-rneΔ10 | derivative of pET15b-rne (Δaa 844-1045) | this work |
| pET15b- | derivative of pET15b-rne (Δaa 585-1045) | this work |
| rneΔCTH | | |

^{a}This notation (aaΔ 844-1045) refers to the amino acids deleted in the protein expressed from the mutant rne allele.

Figures Legends

FIG. 1. A. Primary structure of RNase E. The proline-rich (green), arginine-rich (orange) and glutamic acid-proline-rich (yellow) regions are color coded. The N-terminal half, from residue 1 to 524, is the site of ribonucleolytic activity. This domain is followed by a proline-rich linker (green, 524-568). The central region of RNase E, which is highly charged, contains the arginine-rich RNA binding domain (604-688) that has been shown to bind RNA by Northwestern blotting. The RNA binding domain (RBD) is followed by another proline rich stretch (743-796), (743-796), a second arginine-rich region (796-818, 12 arginines out of 25 residues), which we call AR2, and a third proline-rich region (819-857). The C-terminus includes an acidic region rich in glutamic acid and proline (857-1036) and a C-terminus rich in proline. The ‘protein scaffold’ (688-1061) contains the binding sites for the major components of the RNA degradosome: RhlB, enolase and PNPase. Rh/En (red box) is the region where RhlB and enolase bind to RNase E. The site where PNPase binds is shown by the yellow box.

B. Deletions in the RBD and scaffold of RNase E. The construction of ENS134-1 and ENS134-2, encoding Rne1 and Rne131, was described previously (Iost and Dreyfus, 1995; Lopez et al., 1999). Rne1 is a temperature sensitive enzyme with a glycine (G) to serine (S) substitution at residue 66. Rne131 is encoded by a gene with a +1 frameshift at codon 584. A short 32 amino acid extension is encoded by the +1 reading frame (black box at C-terminal end). In the strains with deletions, the region in RNase E that has been removed is indicated by a thin black line.

FIG. 2. A. The ENS134 strain (Lopez et al., 1994) has the following features. The endogenous lacZ gene has been knocked out but the strain encodes the lac repressor (lacI). The following elements have been inserted into the chromosome: gene 1, encoding bacteriophage T7 RNA polymerase, under the control of a lac promoter (P_{lac}) and a hybrid lacZ gene under the control of a T7 promoter (P_{T7}). The lacZ gene is followed by a small region from the 5′ end of lacY, a tRNA reporter gene and a transcription terminator (Ter). The T7 lacZ message is very sensitive to degradation by RNase (encoded by the rne gene).

B. Degradation of the T7-lacZ mRNA. β-galactosidase levels were measured in the ENS134 derivatives expressing wild type RNase E and the mutant proteins. The strains were grown in M9 medium containing glycerol and casamino acids (0.2% each) with 100 μM IPTG at 30° C. When the cultures reached an OD₆₀₀ of 0.30 the activity was determined as described (Miller, 1972). These results are the average of at least three independent determinations for each strain and the errors bars show the standard deviation.

FIG. 3. A. Western blot analysis of RNase E expression using polyclonal rabbit antibody (upper panel) or MAP antibody (lower panel). The blots were developed using a fluorescence detection system and analyzed by a fluorimager. RNase E (WT) and mutant proteins are indicated at the top of the blots. The asterisks indicate the position of each protein. All the lanes were loaded with equivalent amounts of total protein based on the OD of the cultures except where indicated, i.e. in the first lane 2-fold more WT protein, and 4-fold more RneΔ10 and Rne131.

B. His-tagged RNase E constructs. The black circle to the left signifies the N-terminal HIS tag; the black box to the right, the C-terminal last 16 amino acids, which is conserved in all of these constructs. To the right is indicated the fluorescence signal for each polypeptide (equivalent weights) normalized to the full-length construct.

C. Quantification of the Western blots. The amount of protein relative to wild type is shown for each mutant with the results from the polyclonal antibody in the left column and the MAP antibody at the right. These data represent at least six independent determinations for each mutant and the error bars show the standard deviation.

FIG. 4. A. Gel stained with SYPRO orange showing the separation of extracts containing overexpressed RNase E or the mutant proteins (indicated by the asterisks). pET11a is a control extract with no overexpressed protein. The gel was loaded to give comparable amounts of RNase E and each mutant protein.

B. Blots probed with ³³P-labelled RNA: 5′ UTR of the rne mRNA (upper panel) or 9S ribosomal RNA (lower panel).

FIG. 5. Microcolonies of the P_{lac}-rne131 strain, ENS134(P_{lac}) -2, grown in the presence of 16, 25, 50, 500 and 1000 μM IPTG. Wild type rne under the control of P_{lac} is shown for comparison. Microscope slides were covered with a thin layer (300 μl) of M9 agar containing glycerol and casamino acids (0.2% each). The strains were streaked on these slides and incubated at 37° C. (7h for rne131, 3 h for wild type). Microcolonies were visualized with a Leica DMRB 100× objective and a Coolsnap photometric camera. For the P_{lac}-rne⁺ strain at 16 μM IPTG, we observed two types of colonies: a in which some of the cells were filamented and b where the morphology was normal. For the P_{lac}-rne131 strain at 1000 μM, we observed two types of colonies: a in which the morphology was normal and b where many of the cells formed long filaments.

FIG. 6. Competition during growth between strains encoding RNase E mutant proteins and an isogenic wild type control. The competitor strain is MC1061 (Tet^{S}). The rne mutants and rne⁺ are isogenic derivatives of MC1061 containing a Tn10 transposon (Tet^{R}) linked to the rne loci (AC21 strains). MC1061 was grown overnight in LB. AC21 and its derivatives were grown in LB supplemented with tetracycline (12 μg/ml). AC21 and each derivative were mixed with MC1061 (equal volumes), diluted 10⁻³ in fresh medium lacking the tetracycline, grown at 37° C. until reaching stationary phase, then diluted 10⁻³ in fresh medium lacking tetracycline. By diluting the cultures in the morning and at the end of the afternoon, it is possible to achieve 20 generations of growth per day. Every 20 generations samples were plated on LB agar with or without tetracycline to determine the fraction of cells resistant to tetracycline.

References

Carpousis, A. J., Van Houwe, G., Ehretsmann, C., and Krisch, H. M. (1994) Copurification of E. coli RNAase E and PNPase: evidence for a specific association between two enzymes important in RNA processing and degradation. Cell 76: 889-900.

Carpousis, A. J., Vanzo, N. F., and Raynal, L. C. (1999) mRNA degradation. A tale of poly(A) and multiprotein machines. Trends Genet 15: 24-28.

Casaregola, S., Jacq, A., Laoudj, D., McGurk, G., Margarson, S., Tempete, M., Norris, V., and Holland, I. B. (1992) Cloning and analysis of the entire Escherichia coli ams gene. ams is identical to hmp1 and encodes a 114 kDa protein that migrates as a 180 kDa protein. J Mol Biol 228: 30-40.

Coburn, G. A., and Mackie, G. A. (1999) Degradation of mRNA in Escherichia coli: an old problem with some new twists. Prog Nucleic Acid Res Mol Biol 62: 55-108.

Coburn, G. A., Miao, X., Briant, D. J., and Mackie, G. A. (1999) Reconstitution of a minimal RNA degradosome demonstrates functional coordination between a 3′ exonuclease and a DEAD-box RNA helicase. Genes Dev 13: 2594-2603.

Condon, C., Brechemier-Baey, D., Beltchev, B., Grunberg-Manago, M., and Putzer, H. (2001) Identification of the gene encoding the 5S ribosomal RNA maturase in Bacillus subtilis: mature 5S rRNA is dispensable for ribosome function. Rna 7: 242-253.

Cormack, R. S., and Mackie, G. A. (1992) Structural requirements for the processing of Escherichia coli 5 S ribosomal RNA by RNase E in vitro. J Mol Biol 228: 1078-1090.

Cormack, R. S., Genereaux, J. L., and Mackie, G. A. (1993) RNase E activity is conferred by a single polypeptide: overexpression, purification, and properties of the ams/rne/hmp1 gene product. Proc Natl Acad Sci USA 90: 9006-9010.

Cornet, F., Louarn, J., Patte, J., and Louarn, J. M. (1996) Restriction of the activity of the recombination site dif to a small zone of the Escherichia coli chromosome. Genes Dev 10: 1152-1161.

de la Cruz, J., Kressler, D., Tollervey, D., and Linder, P. (1998) Doblp (Mtr4p) is a putative ATP-dependent RNA helicase required for the 3′ end formation of 5.8S rRNA in Saccharomyces cerevisiae. Embo J 17: 1128-1140.

Deutscher, M. P., and Li, Z. (2001) Exoribonucleases and their multiple roles in RNA metabolism. Prog Nucleic Acid Res Mol Biol 66: 67-105.

Diwa, A., Bricker, A. L., Jain, C., and Belasco, J. G. (2000) An evolutionarily conserved RNA stem-loop functions as a sensor that directs feedback regulation of RNase E gene expression. Genes Dev 14: 1249-1260.

Dziembowski, A., and Stepien, P. P. (2001) Genetic and biochemical approaches for analysis of mitochondrial degradosome from Saccharomyces cerevisiae. Methods Enzymol 342: 367-378.

Ehretsmann, C. P., Carpousis, A. J., and Krisch, H. M. (1992) Specificity of Escherichia coli endoribonuclease RNase E: in vivo and in vitro analysis of mutants in a bacteriophage T4 mRNA processing site. Genes Dev 6: 149-159.

Ghora, B. K., and Apirion, D. (1978) Structural analysis and in vitro processing to p5 rRNA of a 9S RNA molecule isolated from an rne mutant of E. coli. Cell 15: 1055-1066.

Grunberg-Manago, M. (1999) Messenger RNA stability and its role in control of gene expression in bacteria and phages. Annu Rev Genet 33: 193-227.

Iost, I., and Dreyfus, M. (1995) The stability of Escherichia coli lacZ mRNA depends upon the simultaneity of its synthesis and translation. Embo J 14: 3252-3261.

Jacobs, J. S., Anderson, A. R., and Parker, R. P. (1998) The 3′ to 5′ degradation of yeast mRNAs is a general mechanism for mRNA turnover that requires the SKI2 DEVH box protein and 3′ to 5′ exonucleases of the exosome complex. Embo J 17: 1497-1506.

Jager, S., Fuhrmann, O., Heck, C., Hebermehl, M., Schiltz, E., Rauhut, R., and Klug, G. (2001) An mRNA degrading complex in Rhodobacter capsulatus. Nucleic Acids Res 29: 4581-4588.

Jain, C., and Belasco, J. G. (1995) RNase E autoregulates its synthesis by controlling the degradation rate of its own mRNA in Escherichia coli: unusual sensitivity of the rne transcript to RNase E activity. Genes Dev 9: 84-96.

Jain, C., Deana, A., and Belasco, J. G. (2002) Consequences of 5 RNase E scarcity in Escherichia coli. Mol Microbiol 43: 1053-1064.

Jiang, X., Diwa, A., and Belasco, J. G. (2000) Regions of RNase E important for 5′-end-dependent RNA cleavage and autoregulated synthesis. J Bacteriol 182: 2468-2475.

Kaberdin, V. R., Miczak, A., Jakobsen, J. S., Lin-Chao, S., McDowall, K. J., and von Gabain, A. (1998) The endoribonucleolytic N-terminal half of Escherichia coli RNase E is evolutionarily conserved in Synechocystis sp. and other bacteria but not the C-terminal half, which is sufficient for degradosome assembly. Proc Natl Acad Sci USA 95: 11637-11642.

Kido, M., Yamanaka, K., Mitani, T., Niki, H., Ogura, T., and Hiraga, S. (1996) RNase E polypeptides lacking a carboxyl-terminal half suppress a mukB mutation in Escherichia coli. J Bacteriol 178: 3917-3925.

Li, Z., Pandit, S., and Deutscher, M. P. (1999) RNase G (CafA protein) and RNase E are both required for the 5′ maturation of 16S ribosomal RNA. Embo J 18: 2878-2885.

Lin-Chao, S., Wong, T. T., McDowall, K. J., and Cohen, S. N. (1994) Effects of nucleotide sequence on the specificity of rne-dependent and RNase E-mediated cleavages of RNA I encoded by the pBR322 plasmid. J Biol Chem 269: 10797-10803.

Liou, G. G., Jane, W. N., Cohen, S. N., Lin, N. S., and Lin-Chao, S. (2001) RNA degradosomes exist in vivo in Escherichia coli as multicomponent complexes associated with the cytoplasmic membrane via the N-terminal region of ribonuclease E. Proc Natl Acad Sci USA 98: 63-68.

Lopez, P. J., Iost, I., and Dreyfus, M. (1994) The use of a tRNA as a transcriptional reporter: the T7 late promoter is extremely efficient in Escherichia coli but its transcripts are poorly expressed. Nucleic Acids Res 22: 2434.

Lopez, P. J., Marchand, I., Joyce, S. A., and Dreyfus, M. (1999) The C-terminal half of RNase E, which organizes the Escherichia coli degradosome, participates in mRNA degradation but not rRNA processing in vivo. Mol Microbiol 33: 188-199.

Makarova, O. V., Makarov, E. M., Sousa, R., and Dreyfus, M. (1995) Transcribing of Escherichia coli genes with mutant T7 RNA polymerases: stability of lacZ mRNA inversely correlates with polymerase speed. Proc Natl Acad Sci USA 92: 12250-12254.

Margossian, S. P., Li, H., Zassenhaus, H. P., and Butow, R. A. (1996) The DExH box protein Suv3p is a component of a yeast mitochondrial 3′-to-5′ exoribonuclease that suppresses group I intron toxicity. Cell 84: 199-209.

McDowall, K. J., and Cohen, S. N. (1996) The N-terminal domain of the rne gene product has RNase E activity and is non-overlapping with the arginine-rich RNA-binding site. J Mol Biol 255: 349-355.

Miczak, A., Kaberdin, V. R., Wei, C. L., and Lin-Chao, S. (1996) Proteins associated with RNase E in a multicomponent ribonucleolytic complex. Proc Natl Acad Sci USA 93: 3865-3869.

Miller, J. H. (1972) Experiments In Molecular Genetics. Cold Spring Harbor, N.Y.: Cold Spring Harbor Laboratory.

Misra, T. K., and Apirion, D. (1979) RNase E, an RNA processing enzyme from Escherichia coli. J Biol Chem 254: 11154-11159.

Mudd, E. A., Carpousis, A. J., and Krisch, H. M. (1990) Escherichia coli RNase E has a role in the decay of bacteriophage T4 mRNA. Genes Dev 4: 873-881.

Mudd, E. A., and Higgins, C. F. (1993) Escherichia coli endoribonuclease RNase E: autoregulation of expression and site-specific cleavage of mRNA. Mol Microbiol 9: 557-568.

Nogueira, T., de Smit, M., Graffe, M., and Springer, M. (2001) The relationship between translational control and mRNA degradation for the Escherichia coli threonyl-tRNA synthetase gene. J Mol Biol 310: 709-722.

Ow, M. C., Liu, Q., and Kushner, S. R. (2000) Analysis of mRNA decay and rRNA processing in Escherichia coli in the absence of RNase E-based degradosome assembly. Mol Microbiol 38: 854-866.

Py, B., Causton, H., Mudd, E. A., and Higgins, C. F. (1994) A protein complex mediating mRNA degradation in Escherichia coli. Mol Microbiol 14: 717-729.

Py, B., Higgins, C. F., Krisch, H. M., and Carpousis, A. J. (1996) A DEAD-box RNA helicase in the Escherichia coli RNA degradosome. Nature 381: 169-172.

Regnier, P., and Arraiano, C. M. (2000) Degradation of mRNA in bacteria: emergence of ubiquitous features. Bioessays 22: 235-244.

Sambrook, J., and Russell, D. W. (2001) Molecular Cloning. Cold Spring Harbor, N.Y.: Cold Spring Harbor Laboratory.

Schmid, S. R., and Linder, P. (1992) D-E-A-D protein family of putative RNA helicases. Mol Microbiol 6: 283-291.

Sousa, S., Marchand, I., and Dreyfus, M. (2001) Autoregulation allows Escherichia coli RNase E to adjust continuously its synthesis to that of its substrates. Mol Microbiol 42: 867-878.

Steinberg, T. H., Jones, L. J., Haugland, R. P., and Singer, V. L. (1996) SYPRO orange and SYPRO red protein gel stains: one-step fluorescent staining of denaturing gels for detection of nanogram levels of protein. Anal Biochem 239: 223-237.

Symmons, M., Williams, M., Luisi, B., Jones, G. and Carpousis, A. J. (2002) Running rings around RNA: a superfamily of phosphate-dependent RNases. Trends Biochem. Sci., 27: 11-18.

Taraseviciene, L., Bjork, G. R., and Uhlin, B. E. (1995) Evidence for an RNA binding region in the Escherichia coli processing endoribonuclease RNase E. J Biol Chem 270: 26391-26398.

Tock, M. R., Walsh, A. P., Carroll, G., and McDowall, K. J. (2000) The CafA protein required for the 5′-maturation of 16 S rRNA is a 5′-end-dependent ribonuclease that has context-dependent broad sequence specificity. J Biol Chem 275: 8726-8732.

Vanzo, N. F., Li, Y. S., Py, B., Blum, E., Higgins, C. F., Raynal, L. C., Krisch, H. M., and Carpousis, A. J. (1998) Ribonuclease E organizes the protein interactions in the Escherichia coli RNA degradosome. Genes Dev 12: 2770-2781.

1. A process for producing predetermined recombinant polypeptides or proteins, comprising expressing said polypeptides or proteins in Escherichia coli (E. coli) strains whose gene coding RNase E comprises a mutation such that the enzyme produced upon expression of this mutated gene exhibits reduced activity for degrading the messenger RNA (m-RNA) encoding said polypeptides or proteins, compared to bulk cellular mRNA, said mutation not significantly affecting growth of the said E. coli strains, and wherein said mutation corresponds to the substitution or deletion of one up to all the nucleotides located in the region delimited by the nucleotide at position 2193 and the nucleotide at position 2975 of the DNA sequence coding the RNase E represented by SEQ ID NO: 1.

2. A process according to claim 1, characterized in that the mutation causes the deletion of at least one, up to all, of the amino acids at position 585 to 845 of the sequence of RNase E represented by SEQ ID NO: 2.

3. A process according to claim 1, wherein said mutation corresponds to the deletion;
of the DNA fragment delimited by the nucleotides at positions 2193 to 2321 of SEQ ID NO: 1, thus leading to a mutated RNase E gene represented by SEQ ID NO: 3 and coding for the mutated RNase E protein RneΔ24 represented by SEQ ID NO: 4 and corresponding to SEQ ID NO: 2 wherein the sequence delimited by the aminoacids at positions 585 to 627 is deleted,
of the DNA fragment delimited by the nucleotides at positions 2193 to 2519 of SEQ ID NO: 1, thus leading to a mutated RNase E gene represented by SEQ ID NO: 5 and coding for the mutated RNase E protein RneΔ23 represented by SEQ ID NO: 6 and corresponding to SEQ ID NO: 2 wherein the sequence delimited by the aminoacids at positions 585 to 693 is deleted,
of the DNA fragment delimited by the nucleotides at positions 2247 to 2519 of SEQ ID NO: 1, thus leading to a mutated RNase E gene represented by SEQ ID NO: 7 and coding for the mutated RNase E protein RneΔ22 represented by SEQ ID NO: 8 and corresponding to SEQ ID NO: 2 wherein the sequence delimited by the aminoacids at positions 603 to 693 is deleted,
of the DNA fragment delimited by the nucleotides at positions 2247 to 2321 of SEQ ID NO: 1, thus leading to a mutated RNase E gene represented by SEQ ID NO: 9 and coding for the mutated RNase E protein RneΔ21 represented by SEQ ID NO: 4 and corresponding to SEQ ID NO: 2 wherein the sequence delimited by the aminoacids at positions 603 to 627 is deleted,
of the DNA fragment delimited by the nucleotides at positions 2346 to 2519 of SEQ ID NO: 1, thus leading to a mutated RNase E gene represented by SEQ ID NO: 11 and coding for the mutated RNase E protein RneΔ17 represented by SEQ ID NO: 12 and corresponding to SEQ ID NO: 2 wherein the sequence delimited by the aminoacids at positions 636 to 693 is deleted,
of the DNA fragment delimited by the nucleotides at positions 2346 to 2975 of SEQ ID NO: 1, thus leading to a mutated RNase E gene represented by SEQ ID NO: 13 and coding for the mutated RNase E protein RneΔ14 represented by SEQ ID NO: 14 and corresponding to SEQ ID NO: 2 wherein the sequence delimited by the aminoacids at positions 636 to 845 is deleted,
of the DNA fragment delimited by the nucleotides at positions 2622 to 2975 of SEQ ID NO: 1, thus leading to a mutated RNase E gene represented by SEQ ID NO: 15 and coding for the mutated RNase E protein RneΔ18 represented by SEQ ID NO: 16 and corresponding to SEQ ID NO: 2 wherein the sequence delimited by the aminoacids at positions 728 to 845 is deleted.

4. A process according to claim 1, characterized in that the said strains contain an exogenous inducible expression system, under the control of which is placed the expression of the predetermined recombinant polypeptides.

5. A process according to claim 4, wherein the inducible expression system is the expression system using RNA polymerase of the T7 bacteriophage.

6. Process for producing predetermined recombinant polypeptides according to claim 1, characterized in that it comprises:
a step of transforming E. coli strains whose gene coding RNase E comprises a mutation as mentioned in claim 1 such that enzyme produced upon expression of this mutated gene exhibits reduced activity for degrading the messenger RNA (m-RNA) encoding said polypeptides or proteins compared to bulk mRNA, this mutation not significantly affecting growth of the said E. coli strains, with a vector, especially a plasmid, containing the nucleotide sequence coding one or several recombinant polypeptides,
culturing the transformed E. coli strains obtained in the preceding step, for a time sufficient to permit expression of the recombinant polypeptide or polypeptides in the E. coli cells,
and recovery of the recombinant polypeptide or polypeptides produced during the preceding step, optionally after purification of these latter, especially by chromatography, electrophoresis, or selective precipitation.

7. A process according to claim 2, wherein said mutation corresponds to the deletion:
of the DNA fragment delimited by the nucleotides at positions 2193 to 2321 of SEQ ID NO: 1, thus leading to a mutated RNase E gene represented by SEQ ID NO: 3 and coding for the mutated RNase E protein RneΔ24 represented by SEQ ID NO: 4 and corresponding to SEQ ID NO: 2 wherein the sequence delimited by the aminoacids at positions 585 to 627 is deleted,
of the DNA fragment delimited by the nucleotides at positions 2193 to 2519 of SEQ ID NO: 1, thus leading to a mutated RNase E gene represented by SEQ ID NO: 5 and coding for the mutated RNase E protein RneΔ23 represented by SEQ ID NO: 6 and corresponding to SEQ ID NO: 2 wherein the sequence delimited by the aminoacids at positions 585 to 693 is deleted,
of the DNA fragment delimited by the nucleotides at positions 2247 to 2519 of SEQ ID NO: 1, thus leading to a mutated RNase E gene represented by SEQ ID NO: 7 and coding for the mutated RNase E protein RneΔ22 represented by SEQ ID NO: 8 and corresponding to SEQ ID NO: 2 wherein the sequence delimited by the aminoacids at positions 603 to 693 is deleted,
of the DNA fragment delimited by the nucleotides at positions 2247 to 2321 of SEQ ID NO: 1, thus leading to a mutated RNase E gene represented by SEQ ID NO: 9 and coding for the mutated RNase E protein RneΔ21 represented by SEQ ID NO: 4 and corresponding to SEQ ID NO: 2 wherein the sequence delimited by the aminoacids at positions 603 to 627 is deleted,
of the DNA fragment delimited by the nucleotides at positions 2346 to 2519 of SEQ ID NO: 1, thus leading to a mutated RNase E gene represented by SEQ ID NO: 11 and coding for the mutated RNase E protein RneΔ17 represented by SEQ ID NO: 12 and corresponding to SEQ ID NO: 2 wherein the sequence delimited by the aminoacids at positions 636 to 693 is deleted,
of the DNA fragment delimited by the nucleotides at positions 2346 to 2975 of SEQ ID NO: 1, thus leading to a mutated RNase E gene represented by SEQ ID NO: 13 and coding for the mutated RNase E protein RneΔ14 represented by SEQ ID NO: 14 and corresponding to SEQ ID NO: 2 wherein the sequence delimited by the aminoacids at positions 636 to 845 is deleted,
of the DNA fragment delimited by the nucleotides at positions 2622 to 2975 of SEQ ID NO: 1, thus leading to a mutated RNase E gene represented by SEQ ID NO: 15 and coding for the mutated RNase E protein RneΔ18 represented by SEQ ID NO: 16 and corresponding to SEQ ID NO: 2 wherein the sequence delimited by the aminoacids at positions 728 to 845 is deleted.

8. E. coli strains transformed such that they contain an inducible expression system, and whose gene coding RNase E comprises a mutation such that the enzyme produced upon expression of this mutated gene exhibits reduced activity for degrading the messenger RNA (m-RNA) encoding said polypeptides or proteins, compared to bulk mRNA, this mutation not significantly affecting growth of the said E. coli strains, and wherein said mutation corresponds to the deletion:
of the DNA fragment delimited by the nucleotides at positions 2193 to 2321 of SEQ ID NO: 1, thus leading to a mutated RNase E gene represented by SEQ ID NO: 3 and coding for the mutated RNase E protein RneΔ24 represented by SEQ ID NO: 4 and corresponding to SEQ ID NO: 2 wherein the sequence delimited by the aminoacids at positions 585 to 627 is deleted,
of the DNA fragment delimited by the nucleotides at positions 2193 to 2519 of SEQ ID NO: 1, thus leading to a mutated RNase E gene represented by SEQ ID NO: 5 and coding for the mutated RNase E protein RneΔ23 represented by SEQ ID NO: 6 and corresponding to SEQ ID NO: 2 wherein the sequence delimited by the aminoacids at positions 585 to 693 is deleted,
of the DNA fragment delimited by the nucleotides at positions 2247 to 2519 of SEQ ID NO: 1, thus leading to a mutated RNase E gene represented by SEQ ID NO: 7 and coding for the mutated RNase E protein RneΔ22 represented by SEQ ID NO: 8 and corresponding to SEQ ID NO: 2 wherein the sequence delimited by the aminoacids at positions 603 to 693 is deleted,
of the DNA fragment delimited by the nucleotides at positions 2247 to 2321 of SEQ ID NO: 1, thus leading to a mutated RNase E gene represented by SEQ ID NO: 9 and coding for the mutated RNase E protein RneΔ21 represented by SEQ ID NO: 4 and corresponding to SEQ ID NO: 2 wherein the sequence delimited by the aminoacids at positions 603 to 627 is deleted,
of the DNA fragment delimited by the nucleotides at positions 2346 to 2519 of SEQ ID NO: 1, thus leading to a mutated RNase E gene represented by SEQ ID NO: 11 and coding for the mutated RNase E protein RneΔ17 represented by SEQ ID NO: 12 and corresponding to SEQ ID NO: 2 wherein the sequence delimited by the aminoacids at positions 636 to 693 is deleted.

9. E. coli strains according to claim 8, characterized in that the inducible expression system uses RNA polymerase of the T7 bacteriophage.

10. Nucleotide sequence comprising a gene coding RNase E with a mutation such that the enzyme produced upon expression of this mutated gene exhibits reduced activity for degrading the messenger RNA (m-RNA) encoding said polypeptides or proteins, compared to bulk mRNA, said mutation not significantly affecting growth of the said E. coli strains, and wherein said mutation corresponds to the deletion:
of the DNA fragment delimited by the nucleotides at positions 2193 to 2321 of SEQ ID NO: 1, thus leading to a mutated RNase E gene represented by SEQ ID NO: 3 and coding for the mutated RNase E protein RneΔ24 represented by SEQ ID NO: 4 and corresponding to SEQ ID NO: 2 wherein the sequence delimited by the aminoacids at positions 585 to 627 is deleted,
of the DNA fragment delimited by the nucleotides at positions 2193 to 2519 of SEQ ID NO: 1, thus leading to a mutated RNase E gene represented by SEQ ID NO: 5 and coding for the mutated RNase E protein RneΔ23 represented by SEQ ID NO: 6 and corresponding to SEQ ID NO: 2 wherein the sequence delimited by the aminoacids at positions 585 to 693 is deleted,
of the DNA fragment delimited by the nucleotides at positions 2247 to 2519 of SEQ ID NO: 1, thus leading to a mutated RNase E gene represented by SEQ ID NO: 7 and coding for the mutated RNase E protein RneΔ22 represented by SEQ ID NO: 8 and corresponding to SEQ ID NO: 2 wherein the sequence delimited by the aminoacids at positions 603 to 693 is deleted,
of the DNA fragment delimited by the nucleotides at positions 2247 to 2321 of SEQ ID NO: 1, thus leading to a mutated RNase E gene represented by SEQ ID NO: 9 and coding for the mutated RNase E protein RneΔ21 represented by SEQ ID NO: 4 and corresponding to SEQ ID NO: 2 wherein the sequence delimited by the aminoacids at positions 603 to 627 is deleted,
of the DNA fragment delimited by the nucleotides at positions 2346 to 2519 of SEQ ID NO: 1, thus leading to a mutated RNase E gene represented by SEQ ID NO: 11 and coding for the mutated RNase E protein RneΔ17 represented by SEQ ID NO: 12 and corresponding to SEQ ID NO: 2 wherein the sequence delimited by the aminoacids at positions 636 to 693 is deleted.
